SEQUENCE LISTING (54 sequences)

SEQ ID NO: 1 - protein, 179 aa, Homo sapiens
  SHH protein
MLSLFPSPGPGSSRCKDKLNALAISVMNQWPGVKLRVTEGWDEDGHHSEESLHYEGRAVDITTSDRDRSKYGMLARLAVEAGFDWVYYESKAHIHCSVKAGKERKPLLRDTRYRASRGCSPAPPGRGADFSFRVDGAKSLCKMYAWWLLPASLMTKCISIPVKTCITMYLKLLSLCSIV

SEQ ID NO: 2 - DNA, 828 bp, Homo sapiens
  SHH sequence
agacacgctctccccgcgcgggcctaggtgccaggcgagggtgctggcggccagggggctcctaaggggcaggaggccagagggccggatctgaagcctggagtggggtcccgagccgctacactaaatagatttaatgtgcgctctggggccgccaggaaagacgctcaggcctcgctaggagcatcggctgtttcaggacctggagaaaggcccccagctctaccctgagaggacgtgctcctccacgctcctccgcaaatgctgtccctcttccccagcccagggcccggctcttcgaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcaggcaaggaaaggaaaccactgcttagagacactaggtacagggcatcaagagggtgcagcccagccccgccaggacgaggcgccgacttctcattcagggttgacggagccaagagtttgtgcaaaatgtatgcatggtggttgctgccagcctccctcatgactaaatgcatatctattcctgtcaaaacgtgtatcacaatgtacttgaaactgttatctttgtgctctattgtttgaataattaaagaaattacagagactctc

SEQ ID NO: 3 - protein, 455 aa, Homo sapiens
  BMP4 protein
MREGRGGGREGRSAEPGPEARSHSVVPSRATHCCSFPEPFQQVCSRLAVKNHGLLLYALFSVILLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 4 - protein, 345 aa, Homo sapiens
  BMP4 protein
MFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 5 - protein, 345 aa, Homo sapiens; identical to SEQ ID NO: 4
  BMP4 protein

SEQ ID NO: 6 - protein, 345 aa, Homo sapiens; identical to SEQ ID NO: 4
  BMP4 protein

SEQ ID NO: 7 - DNA, 1715 bp, Homo sapiens
  BMP4 sequence
ggaggggccgccggggaagaggaggaggaaggaaagaaagaaagcgagggagggaaagaggaggaaggaagatgcgagaaggcagaggaggagggagggagggaaggagcgcggagcccggcccggaagctaggagccattccgtagtgccatcccgagcaacgcactgctgcagcttccctgagcctttccagcaagtttgttcaagattggctgtcaagaatcatggactgttattatatgccttgttttctgtcatcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggttggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaa

SEQ ID NO: 8 - DNA, 1944 bp, Homo sapiens
  BMP4 sequence
ggaggggccgccggggaagaggaggaggaaggaaagaaagaaagcgagggagggaaagaggaggaaggaagatgcgagaaggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggagccattccgtagtgccatcccgagcaacgcactgctgcagcttccctgagcctttccagcaagtttgttcaagattggctgtcaagaatcatggactgttattatatgccttgttttctgtcatcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaa

SEQ ID NO: 9 - DNA, 1640 bp, Homo sapiens
  BMP4 sequence
gtgactccgaggggctggaagaaaaacagagcctgtctgcggtggagtctcattatattcaaatattccttttaggagccattccgtagtgccatcccgagcaacgcactgctgcagcttccctgagcctttccagcaagtttgttcaagattggctgtcaagaatcatggactgttattatatgccttgttttctgtcatcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaagttatatttataactacgtattc

SEQ ID NO: 10 - DNA, 2027 bp, Homo sapiens
  BMP4 sequence
gggcgcccgcggcctccgcacccggacctgaggtgttggtcgactccgggcatccacggtcgggagggagggctgagctgttcgatcctttacttttcttcctcaaagtctacctgccaatgcccctaagaagaaaaccaagtatgtgcgtggagagtggggcggcaggcaacccgagttcttgagctccggagcgacccaaagcagcaactgggaacagcctcaggaaagggaggtcgggtggagtgggctttggggcaggagtcatggggcccgggccccggggacgacctggcgctcccggccctgctgaacgctgagttgcgcctagtcgggttttcgaagaggcccttgcgcagagcgacccacgcgcgcggcagcatcttcgattagtcaggacatcccagtaactgcttgaactgtaggagccattccgtagtgccatcccgagcaacgcactgctgcagcttccctgagcctttccagcaagtttgttcaagattggctgtcaagaatcatggactgttattatatgccttgttttctgtcatcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaa

SEQ ID NO: 11 - DNA, 30 bp, Artificial Sequence
  Shh promotor F(Mouse)
tttttagttttgttattatttaaaattagg

SEQ ID NO: 12 - DNA, 25 bp, Artificial Sequence
  Shh promotor R(Mouse)
caaaaatcaccaaaaaacatctaac

SEQ ID NO: 13 - DNA, 25 bp, Artificial Sequence
  Shh upshore region 1 F(Mouse)
tttgtatatttatatttggggatgg

SEQ ID NO: 14 - DNA, 28 bp, Artificial Sequence
  Shh upshore region 1 R(Mouse)
aaaaaacttataaaacaaactacctttc

SEQ ID NO: 15 - DNA, 27 bp, Artificial Sequence
  Shh upshore region 2 F(Mouse)
ttgtattttgttaggatagattggaag

SEQ ID NO: 16 - DNA, 25 bp, Artificial Sequence
  Shh upshore region 2 R(Mouse)
accccatccccaaatataaatatac

SEQ ID NO: 17 - DNA, 25 bp, Artificial Sequence
  Shh upshore region 3 F(Mouse)
ggatggtgaggttttgttatattgt

SEQ ID NO: 18 - DNA, 25 bp, Artificial Sequence
  Shh upshore region 3 R(Mouse)
ggatggtgaggttttgttatattgt

SEQ ID NO: 19 - DNA, 26 bp, Artificial Sequence
  Shh upshore region 4 F(Mouse)
tgaagtaaaatgaggttttaggatgt

SEQ ID NO: 20 - DNA, 25 bp, Artificial Sequence
  Shh upshore region 4 R(Mouse)
caccatcccaaacttaaaaaaatta

SEQ ID NO: 21 - DNA, 25 bp, Artificial Sequence
  Shh downshore region 1 F(Mouse)
atgttgttgttgttggttagatgtt

SEQ ID NO: 22 - DNA, 25 bp, Artificial Sequence
  Shh downshore region 1 R(Mouse)
ataaaaaaccccatcttctaatacc

SEQ ID NO: 23 - DNA, 25 bp, Artificial Sequence
  Shh downshore region 2 F(Mouse)
gggtattagaagatggggtttttta

SEQ ID NO: 24 - DNA, 25 bp, Artificial Sequence
  Shh downshore region 2 R(Mouse)
cccaaactttctcaattacaattct

SEQ ID NO: 25 - DNA, 24 bp, Artificial Sequence
  Shh downshore region 3 F(Mouse)
gaaagtttgggggtagttttgata

SEQ ID NO: 26 - DNA, 25 bp, Artificial Sequence
  Shh downshore region 3 R(Mouse)
tatttacaaaaaaacccatttccaa

SEQ ID NO: 27 - DNA, 25 bp, Artificial Sequence
  Shh promotor F(Human)
tttttttgttttttgattgttgttt

SEQ ID NO: 28 - DNA, 26 bp, Artificial Sequence
  Shh promotor R(Human)
tcaactttttaaaatacctcctcttc

SEQ ID NO: 29 - DNA, 25 bp, Artificial Sequence
  Shh upshore region 1 F(Human)
ttttggggaagaaaaattaaataat

SEQ ID NO: 30 - DNA, 27 bp, Artificial Sequence
  Shh upshore region 1 R(Human)
caacaatcaaaaaacaaaaaaaatcta

SEQ ID NO: 31 - DNA, 26 bp, Artificial Sequence
  Shh upshore region 2 F(Human)
agtgaggtgattatagatttaaagat

SEQ ID NO: 32 - DNA, 26 bp, Artificial Sequence
  Shh upshore region 2 R(Human)
caactattatttaatttttcttcccc

SEQ ID NO: 33 - DNA, 25 bp, Artificial Sequence
  Shh upshore region 3 F(Human)
atttgtaaagggaatttttggaaat

SEQ ID NO: 34 - DNA, 27 bp, Artificial Sequence
  Shh upshore region 3 R(Human)
aaccaaaaaaataaaatttaaaactcc

SEQ ID NO: 35 - DNA, 25 bp, Artificial Sequence
  Shh upshore region 4 F(Human)
tgttaagggtggaaggtagggtagt

SEQ ID NO: 36 - DNA, 25 bp, Artificial Sequence
  Shh upshore region 4 R(Human)
caaaaattccctttacaaatcaact

SEQ ID NO: 37 - DNA, 26 bp, Artificial Sequence
  Shh downshore region 1 F(Human)
ggaagaggaggtattttaaaaagttg

SEQ ID NO: 38 - DNA, 25 bp, Artificial Sequence
  Shh downshore region 1 R(Human)
aactaaacccttaacctccattctc

SEQ ID NO: 39 - DNA, 25 bp, Artificial Sequence
  Shh downshore region 2 F(Human)
gagaatggaggttaagggtttagtt

SEQ ID NO: 40 - DNA, 24 bp, Artificial Sequence
  Shh downshore region 2 R(Human)
cctcctaacttttccaattaaaaa

SEQ ID NO: 41 - DNA, 25 bp, Artificial Sequence
  Shh downshore region 3 F(Human)
atttttaattggaaaagttaggagg

SEQ ID NO: 42 - DNA, 25 bp, Artificial Sequence
  Shh downshore region 3 R(Human)
caaaaaaacccatttctaacttcaa

SEQ ID NO: 43 - DNA, 21 bp, Mus musculus
  Bmpr1a target sequence
ctttagcctacaagcagttta

SEQ ID NO: 44 - RNA, 21 nt, Artificial Sequence
  Bmpr1a sense sequence(Mouse)
gggucguuacaaccgugauuu

SEQ ID NO: 45 - RNA, 21 nt, Artificial Sequence
  Bmpr1a antisense sequence(Mouse)
aaaucacgguuguaacgaccc

SEQ ID NO: 46 - DNA, 21 bp, Mus musculus
  Shh target sequence
ctttagcctacaagcagttta

SEQ ID NO: 47 - RNA, 21 nt, Artificial Sequence
  Shh sense sequence(Mouse)
cuuuagccuacaagcaguuua

SEQ ID NO: 48 - RNA, 21 nt, Artificial Sequence
  Shh antisense sequence(Mouse)
uaaacugcuuguaggcuaaag

SEQ ID NO: 49 - DNA, 21 bp, Homo sapiens
  Bmpr1a target sequence
gtccagatgatgctattaata

SEQ ID NO: 50 - RNA, 21 nt, Artificial Sequence
  Bmpr1a sense sequence(Human)
guccagaugaugcuauuaaua

SEQ ID NO: 51 - RNA, 21 nt, Artificial Sequence
  Bmpr1a antisense sequence(Human)
uauuaauagcaucaucuggac

SEQ ID NO: 52 - DNA, 21 bp, Homo sapiens
  Shh target sequence
ctacgagtccaaggcacatat

SEQ ID NO: 53 - RNA, 21 nt, Artificial Sequence
  Shh sense sequence(Human)
cuacgaguccaaggcacauau

SEQ ID NO: 54 - RNA, 25 nt, Artificial Sequence
  Shh antisense sequence(Human)
atttttaattggaaaagttaggagg

(19) United States
(12) Patent Application Publication
(10) Pub. No.: US 2021/0060044 A1
(43) Pub. Date: Mar. 4, 2021

(54) METHODS FOR TREATING BLADDER CANCER BY ACTIVATION OF HEDGEHOG SIGNALING USING A METHYLATION INHIBITOR

(71) Applicant: POSTECH Research and Business Development Foundation, Pohang-si (KR)

(72) Inventors: Kunyoo SHIN, Pohang-si (KR); SungEun KIM, Seoul (KR); Eunjee KIM, Seoul (KR)

(21) Appl. No.: 16/993,447

(22) Filed: Aug. 14, 2020

(30) Foreign Application Priority Data
Aug. 26, 2019 (KR) 10-2019-0104753

(51) Int. Cl. A61K 31/706; A61P 35/00; C12Q 1/6869

(52) U.S. Cl. CPC A61K 31/706; C12Q 2600/154; C12Q 1/6869; A61P 35/00

(57) ABSTRACT

The present invention relates to a composition for treating bladder cancer, which includes a methylation inhibitor of the Sonic hedgehog (SHH) gene, and more particularly, to a composition for preventing or treating bladder cancer, which includes a methylation inhibitor as an active ingredient to activate a hedgehog (Hh) signaling pathway involving a protein encoded by the gene by maintaining the expression level of SHH gene by suppressing methylation at the specific sites of the promoter of the SHH gene.

The inventors found that the methylation at specific sites of the promoter of the SHH gene changes the pattern of the gene expression, and first identified that bladder cancer can be prevented or treated by controlling the Hh signaling pathway involving a protein encoded by the gene. Therefore, since the growth of cancer cells may be inhibited by inducing differentiation of the bladder cancer cells to a luminal subtype by activating the Hh signaling pathway by suppressing the methylation of the promoter of the SHH gene, the composition according to the present invention is expected to be effectively used in the treatment of bladder cancer.

### CROSS-REFERENCE TO RELATED APPLICATION

This application claims priority to and the benefit of Korean Patent Application No. 10-2019-0104753, filed on Aug. 26, 2019, the disclosure of which is incorporated herein by reference in its entirety.

### TECHNICAL FIELD

The present invention relates to a composition for treating bladder cancer, which includes a methylation inhibitor for the Sonic hedgehog (SHH) gene, and more particularly, to a composition for preventing or treating bladder cancer, which includes, as an active ingredient, a methylation inhibitor that activates a hedgehog (Hh) signaling pathway involving a protein encoded by the gene by inhibiting methylation at a specific site of the promoter of the SHH gene to maintain an expression level of the SHH gene.

### BACKGROUND ART

Bladder cancer is a malignant tumor that occurs in the bladder. Most of the bladder cancers are epithelial tumors derived from epithelial cells, and malignant epithelial tumors include transitional epithelial cell carcinoma (urothelial carcinoma), squamous cell carcinoma and adenocarcinoma, sarcomas derived from muscles of the bladder, small cell carcinoma derived from nerve cells, malignant lymphoma, and metastatic cancer of the bladder in which cancer from other organs has spread to the bladder.

Sonic hedgehog (SHH) is a protein encoded by the SHH gene, and both of the SHH gene and protein may be denoted SHH. SHH is one of three proteins in the mammalian signaling pathway family called hedgehog. Another one of the proteins constituting the family is Desert hedgehog (DHH), and the other is Indian hedgehog (IHH).

The hedgehog (Hh) signaling pathway is a signaling pathway transmitting information required for cell differentiation to embryonic cells. In different parts of an embryo, different concentrations of the hedgehog protein are contained, and it is known that a mouse in which a gene related to the protein is knocked out has a brain, skeleton, muscles, gastrointestinal tract and lungs, which are not properly developed.

Meanwhile, in recent research, it was reported that the Hh signaling pathway is related to the regulation of adult stem cells involved in the maintenance and renewal of adult tissue, and also related to the onset of some types of cancer (Ther Adv Med Oncol. 2010 Jul.; 2(4): 237-250, Naoko Takebe). However, there is no research on the prevention or treatment of bladder cancer using the same.

### DISCLOSURE

### Technical Problem

Therefore, the inventors had made an earnest effort to study the use of an interaction between cancer cells and tumor stroma in treatment of bladder cancer, finding that methylation at specific sites of the promoter of the Sonic hedgehog (SHH) gene changes an expression pattern of the gene, and first identifying that bladder cancer can be prevented or treated by regulating a signaling pathway involving a protein encoded by the gene. Based on this, the present invention was completed.

The present invention is directed to providing a composition for preventing or treating bladder cancer, which includes a methylation inhibitor of the SHH gene as an active ingredient.

The present invention is also directed to providing a method of screening a material for treating bladder cancer.

The present invention is also directed to providing a composition for diagnosing bladder cancer, which includes an agent for measuring a methylation level of the SHH gene.

The present invention is also directed to providing an in vitro composition for inducing the conversion of a basal subtype of bladder cancer cells to a luminal subtype.

However, technical problems to be solved in the present invention are not limited to the above-described problems, and other problems which are not described herein will be fully understood by those of ordinary skill in the art from the following descriptions.

### Technical Solution

To attain the objects of the present invention, the present invention provides a composition for preventing or treating bladder cancer, which includes a methylation inhibitor of the SHH gene as an active ingredient.

In one embodiment of the present invention, the methylation inhibitor may inhibit methylation in the promoter region of the SHH gene.

In another embodiment of the present invention, the promoter region may be a 2kb-upstream region of a CpG island.

In still another embodiment of the present invention, the methylation inhibitor may be 5′-azacitidine.

In yet another embodiment of the present invention, the composition may increase BMP4 expression.

In yet another embodiment of the present invention, the composition may inhibit the growth of bladder cancer cells.

In addition, the present invention provides a method of screening a material for treating bladder cancer, which includes the following steps:

(a) treating a biological sample derived from a subject with a candidate material;

(b) measuring a methylation level of the SHH gene in the sample treated with the candidate material; and

(c) selecting the sample as a material for treating bladder cancer when the methylation level of the SHH gene decreases, compared with a control not treated with a candidate material.

In one embodiment of the present invention, the candidate material may be selected from the group consisting of a compound, a microbial culture solution or extract, a natural substance extract, a nucleic acid and a peptide.

In addition, the present invention provides a composition for diagnosing bladder cancer, which includes an agent for measuring a methylation level of the SHH gene.

In addition, the present invention provides an in vitro composition for inducing conversion of a basal subtype of bladder cancer cells to a luminal subtype.

In one embodiment of the present invention, the composition may decrease the expression of a basal subtype-specific marker in bladder cancer cells, and increase the expression of a luminal subtype-specific marker.

In another embodiment of the present invention, the luminal subtype-specific marker may be any one or more selected from the group consisting of KRT18, UPK1B, FOXA1, KRT20, GATA3, PPARG, UPK3A, UPK2, UPK1A and Ck18.

In addition, the present invention provides a method for preventing or treating bladder cancer, which includes administering the composition into a subject.

In addition, the present invention provides a use of the composition for preventing or treating bladder cancer.

### Advantageous Effects

The inventors found that methylation at specific sites of the promoter of

Sonic Hedgehog (SHH) gene changes an expression pattern of the gene, and bladder cancer can be prevented or treated by regulating a Hh signaling pathway involving a protein encoded by the gene, and the distribution of bladder cancer cells to a luminal subtype can be induced by activating a Hh signaling pathway through the inhibition of methylation of the promoter for the SHH gene to inhibit the growth of cancer cells. The composition according to the present invention is expected to be effectively used in the treatment of bladder cancer.

### DESCRIPTION OF DRAWINGS

The above and other objects, features and advantages of the present invention will become more apparent to those of ordinary skill in the art by describing in detail exemplary embodiments thereof with reference to the accompanying drawings, in which:

FIGS. 1A to 1F show the effect of 5′-azacitidine on the methylation of the Shh gene, confirming that a DNA methylation level is reduced in the CpG shore upstream of the Shh promoter region of a mouse in which BBN-induced urothelial carcinoma occurs (the average degree of methylation is indicated by the black portion of a white circle) (FIG. 1A); the summary of results obtained from bisulfite sequencing analysis of the BBN-induced urothelial carcinoma mouse (FIG. 1B); results showing that Shh expression significantly increases when the methylation level is reduced as described above (FIG. 1C); results showing that the methylation in the promoter region of the Shh gene of the rodent increases to confirm whether the loss of the Shh expression is caused by the methylation of the Shh gene (FIG. 1D); the summary of results obtained by bisulfite sequencing analysis of the bladder organoids (FIG. 1E); and results showing that a methylation level is reduced after 5′-azacitidine treatment (FIG. 1F).

FIGS. 2A to 2E show results illustrating the inhibition of bladder cancer development in an early stage of tumor development by inhibiting DNA methylation by 5′-azacitidine treatment and its inhibition mechanism, experimental groups were divided into i) a group treated only with BBN without 5′-azacitidine treatment for 6 months and ii) a group treated with a low dose of 5′-azacitidine for 2 months from month 4 after the BBN treatment (FIG. 2A); results obtained by H&E staining to confirm the expression of invasive carcinoma confirmed in each experimental group (Scale bars represent 150 μm) (FIG. 2B); schematic diagrams of experiments for continuous exposure of mice in which a stromal Hh response is genetically inhibited to BBN additionally for 2 months in the presence of 5′-azacitidine (FIG. 2B); and results obtained from H&E staining to confirm that the anticancer initiation effect of 5′-azacitidine disappears in mice in which a stromal Hh response is genetically inhibited (Scale bar represent 300 μm) (FIGS. 2C to 2E).

FIGS. 3A to 3K show results confirming that 5′-azacitidine treatment inhibits DNA methylation, thereby suppressing the growth of mature urothelial carcinoma, and showing the mechanism of inhibition, wherein BBN-induced tumor cells derived from allogeneic mice are orthotopically injected into mice, and then experimental groups are divided into i) a group not treated with 5′-azacitidine and ii) a group treated with 5′-azacitidine for 1.5 months (FIG. 3A); H&E staining results of the expression of invasive carcinoma for each experimental group (FIG. 3B); schematic diagrams of experiments for treating models prepared by implanting allogeneic mouse-derived tumors into mice in which a stromal Hh response is genetically inhibited with 5′-azacitidine (FIG. 3C); results obtained by H&E staining to confirm that the anticancer propagation effect of 5′-azacitidine disappears in mouse models in which a stromal Hh response is genetically inhibited (Scale bars represent 150 μm) (FIGS. 3D and 3E); schematic diagrams of experiments for overexpressing Bmp4 in organoids, genetically removing a stromal Hh signaling pathway, and using mice treated with 5′-azacitidine for one month to increase Shh expression (FIG. 3F); results obtained from H&E staining to confirm that the growth of tumor organoids is reduced in the mice (Scale bars represent 300 μm) (FIGS. 3G and 3H); and results obtained by culturing tumor organoids derived from BBN-induced bladder tumors in the absence or presence of Bmp4 for 8 days (Scale bars represent 100 μm) (FIG. 3I); the average size of bladder tumor organoids cultured for 4,6, and 8 days in the absence or presence of the Bmp4 protein (FIG. 3J); and quantification results of cell proliferation in tumor organoids cultured for 6 days in the absence or presence of Bmp4 (FIG. 3K).

FIGS. 4A to 4G show results illustrating the effect of 5′-azacitidine on subtype differentiation of urothelial carcinoma cells: schematic diagrams of experiments for evaluating the effect of DNA methyltransferase inhibition on the growth of bladder cancer under immunocompromised conditions (FIG. 4A); H&E staining of allografts in a vehicle control and magnification results thereof (Scale bars represent 150 μm) (FIG. 4B); H&E staining of allografts treated with 5′-azacitidine and magnification results thereof (Scale bars represent 150 μm) (FIG. 4C); results showing that the expression of a basal marker increases in allografts in a vehicle control (represented in green) (FIG. 4D); results showing that the expression of a luminal marker increases in allografts treated with 5′-azacitidine (represented in red) (FIG. 4E); results confirming the increase in expression of luminal markers (Upk1a, Upk1b, Upk2, Upk3a, Upk3b, Krt20, and Krt18) in allografts treated with 5′-azacitidine, compared with a vehicle control (normalized to a basal marker Krt5) (FIG. 4F); and results obtained from gene set enrichment analysis (GSEA) of tumor allografts treated with a vehicle control and 5′-azacitidine from RNA-Seq data using conventional standard luminal and basal signatures (FIG. 4G).

FIGS. 5A to 5H show results illustrating the association of Hh and Bmp signaling feedback between tumor and stroma in subtype conversion of bladder cancer cells: schematic diagram of an experiment for orthotopic transplantation of BBN-induced tumor organoids expressing shRNA targeting Shh (FIG. 5A); results obtained from H&E staining of control tumor organoids, tumor organoids expressing shRNA targeting Shh, and tumor organoids expressing shRNA targeting Bmpr1a (Ck5 is represented in green, Ck18 is represented in red, and Scale bars represent 100 μm) (FIG. 5B); results confirming expression levels of Upk1a, Upk2, Upk3a and Krt18 in control organoids, tumor organoids expressing shRNA targeting Shh, and tumor organoids expressing shRNA targeting Bmpr1a (FIG. 5C); results obtained from GSEA in tumor organoid allografts expressing shRNA targeting Shh from RNA-Seq data using a conventional standard luminal signature (FIG. 5D); results obtained from GSEA of tumor organoid allografts expressing shRNA targeting Bmpr1a (FIG. 5E); a schematic diagram of an experiment for transplanting a mixture of tumor organoids expressing shRNA targeting Shh, which are labeled with mCherry, tumor organoids expressing shRNA targeting Bmpr1a, which are labeled with mCherry, control organoids labeled with EGFP into microenvironments of the same living animals (FIG. 5F); results obtained from H&E staining and immunostaining to confirm that when allografts are treated with 5′-azacitidine, organoids expressing shRNA targeting Shh, which are labeled with mCherry, develop to the more aggressive and rapidly growing basal-like subtype, whereas an EGFP-labeled tumor develops to the less aggressive luminal-like subtype (FIG. 5G); and results obtained from H&E staining and immunostaining to confirm that, when a mixture of tumor organoids expressing shRNA targeting Bmpr1a, which are labeled with mCherry, and EGFP-labeled control organoids is transplanted into microenvironments of the same living animals, and the animals are treated with 5′-azacitidine, mCherry-labeled tumor organoids develop to the more aggressive and rapidly growing basal-like subtype, whereas EGFP-labeled tumors develop to the less aggressive luminal-like subtype (Scale bars represent 50 μm) (FIG. 5H).

FIGS. 6A to 6I show that increased methylation of the SHH gene induces the basal subtype of human urothelial carcinoma through decreased activity of Hh/BMP signaling feedback between cancer cells and tumor stroma: results obtained from bisulfite sequencing to confirm that the methylation of the CpG shore of the SHH gene promoter region significantly increases in human muscle invasive bladder cancer cell lines J82, T24 and TCC-SUP (each circle represents one of 117 CpG sites, and the average degree of methylation is indicated by the black part of a white circle) (FIG. 6A); the summary of the bisulfite sequencing analysis results (FIG. 6B); result showing that the SHH expression levels in J82, T24 and TCC-SUP treated with 5′-azacitidine are increased (FIG. 6C); establishment of orthotopic xenograft models treated with 5′-azacitidine for one month, following transplantation of a human muscle invasive bladder cancer cell line J82 into immunocompromised mice (Nod/Scid/Rag2) (FIG. 6D); H&E staining and magnification results of mouse xenografts treated with the vehicle control or 5′-azacitidine (Scale bars represent 300 μm) (FIG. 6E); results confirming the expression levels of luminal markers (FOXA1 and GATA3) and basal markers (CDH3 and KRT6A) in tumor xenografts from mice treated with 5′-azacitidine, compared with those of the vehicle control (FIG. 6F); the schematic diagram of an experiment for orthotopically xenografting a cell line expressing shRNA targeting SHH or BMPR1A, and treating the mice with 5′-azacitidine for one month (FIG. 6G); results confirming the expression of shRNA targeting SHH or BMPR1A in the mice (FIG. 6H); and results confirming the expression levels of luminal markers (FOXA1 and GATA3) and basal markers (CDH3 and KRT6A) in tumor xenografts injected with a control, J82 expressing shRNA targeting SHH or BMPR1A (FIG. 6I).

FIGS. 7A to 7D show results of patient-derived urothelial carcinomas and large-scale transcriptional analyses: results of comparing the expression levels of basal markers (KRT5, KRT14, CD44 and KRT6A) and luminal markers (UPK1A, UPK2, ERBB2, FOXA1 and GATA3) in 10 patients (FIG. 7A); results showing SHH expression (white) and two subtypes of invasive urothelial carcinoma (basal; dark grey, luminal; light grey) in benign urothelium from patients (FIG. 7B); results of analyzing the methylation status of the human SHH gene in human invasive urothelial carcinoma tissue derived from patients (three benign tissues, six basal tumors and three luminal tumors) (FIG. 7C); and the summary of the analysis results (FIG. 7D).

### MODES OF THE INVENTION

Hereinafter, the present invention will be described in further detail.

The inventors found that methylation at specific sites of the promoter of Sonic Hedgehog (SHH) gene changes an expression pattern of the gene, identified a Hh signaling pathway involving a protein encoded by the gene, first confirming that bladder cancer can be prevented or treated by regulating the signaling pathway. Thus, the present invention was completed.

Therefore, one aspect of the present invention provides a composition for preventing or treating bladder cancer, which includes a methylation inhibitor of the SHH gene as an active ingredient.

The “bladder cancer,” which is a disease indicated herein is a malignant tumor occurring in the bladder. Most of the bladder cancers are epithelial tumors derived from epithelial cells, and malignant epithelial tumors include transitional epithelial cell carcinoma (urothelial carcinoma), squamous cell carcinoma and adenocarcinoma, sarcomas derived from muscles of the bladder, small cell carcinoma derived from nerve cells, malignant lymphoma, and metastatic cancer of the bladder in which cancer from other organs has spread to the bladder.

More specifically, transitional epithelial cell carcinoma (urothelial carcinoma) is derived from urothelial cells that come into direct contact with urine, accounts for most cases of bladder cancer, and may occur in the upper urinary tract, including the renal pelvis and the ureter, as well as the bladder. Transitional epithelial cell carcinomas (urothelial carcinomas) are classified into three grades according to the degree of cell differentiation (degree of cell migration). In 1973, the World Health Organization (WHO) defined good differentiation (grade 1) as the degree of differentiation is closest to normal, poor differentiation (grade 3) which is opposite to grade 1, and average differentiation (grade 2) which is not included in either of these. It is known that, in grades 1 through 3, 6%, 52% and 82% or more of tumors are the submucosal invasion type, respectively. In addition, squamous cell carcinoma accounts for approximately 3% of bladder cancer cases, is common in men, usually has high malignancy and invasiveness, and it is known that squamous cell carcinoma occurs in patients with spinal cord injury, who carry a urinary catheter consistently, patients with chronic bladder mucosal irritation by bacterial infection or foreign matter (bladder stones) in the bladder, or patients with chronic urinary disorder symptoms.

Bladder cancer may be classified into, according to stages of progression, non-muscle-invasive (superficial) bladder cancer that can be completely removed by transurethral resection because tumors are confined to only the bladder mucosa or submucosal layer, muscle-invasive bladder cancer that requires bladder resection for completely removal of tumors because the bladder cancer has invaded a muscle layer, and metastatic bladder cancer. Approximately 70% of bladder cancer cases is diagnosed as non-muscle-invasive (superficial) bladder cancer, which protrudes from inside the bladder in a cabbage or sea anemone shape, does not metastasize easily, but recurs after surgery in almost all cases and can develop to muscle-invasive bladder cancer. In addition, in the present invention, the bladder cancer may be non-muscle-invasive (superficial) bladder cancer, muscle-invasive bladder cancer, or metastatic cancer, and preferably, muscle-invasive bladder cancer, but the present invention is not limited thereto.

The “Sonic hedgehog (SHH)” is the most widely studied ligand in the hedgehog signaling pathway, and the ligand is known to play a critical role in regulation of organogenesis in vertebrates, such as the growth of the number of limbs and the development of brain tissue during the development of an individual, and reported to regulate cell division of adult stem cells in an adult and is associated with the development of some types of cancer.

In addition, the term “Sonic hedgehog (Shh)” is a mammalian homologous protein of mouse SHH.

A Sonic hedgehog (SHH) protein and a gene encoding the protein according to the present invention may be selected from amino acid sequence data or base sequence data of human-derived SHH, or derived from a mouse, the SHH protein preferably consists of an amino acid sequence of SEQ ID NO: 1 (NCBI accession number: NP_001297391.1), and the gene encoding the protein may consist of a base sequence of SEQ ID NO: 2 (NCBI accession number: NM_001310462.2), but the present invention is not limited thereto.

A BMP4 protein and a gene encoding the protein according to the present invention may be one or more selected from the amino acid sequence data and base sequence data of human-derived BMP4, or derived from a mouse, the BMP4 protein preferably consists of an amino acid sequence of SEQ ID NO: 3 (NCBI accession number: NP_001334841.1), SEQ ID NO: 4 (NCBI accession number: NP_001334842.1), SEQ ID NO: 5 (NCBI accession number: NP_001334844.1) or SEQ ID NO: 6 (NCBI accession number: NP_001334846.1), and the gene encoding the protein preferably consists of a base sequence of SEQ ID NO: 7 (NCBI accession number: NM_001347912.1), SEQ ID NO: 8 (NCBI accession number: NM_001347913.1), SEQ ID NO: 9 (NCBI accession number: NM_001347915.1) or SEQ ID NO: 10 (NCBI accession number: NM_001347917.1), but the present invention is not limited thereto.

The “regulation of a hedgehog (Hh) signaling pathway” is reported to be associated with the secretion, absorption and translocation of the ligand “SHH protein.”

In the present invention, the methylation inhibitor may inhibit methylation of the promoter region of the SHH gene, and the promoter region may be a 2kb-upstreamregion of a CpG island, and the level of methylation of the region of the CpG island may be inhibited to 36% or more and 43% or less, and the methylation inhibitor may be 5′-azacitidine, but the present invention is not limited thereto.

In the present invention, the composition may also increase BMP4 expression, and inhibit the growth of bladder cancer cells.

In addition, the present invention provides an in vitro composition for inducing subtype conversion of bladder cancer cells, which includes the methylation inhibitor, and the composition may increase the expression of a luminal subtype-specific marker in the bladder cancer cells. The luminal subtype-specific marker may be KRT18, UPK1B, FOXA1, KRT20, GATA3, PPARG, UPK3A, UPK2 or UPK1A for a human, or Krt18, Upk1b, Foxa1, Krt20, Gata3, Upk3a, Upk2, Upk1a or Upk3b for a mouse, but the present invention is not limited thereto.

The term “prevention” used herein refers to all actions of inhibiting bladder cancer or delaying the onset thereof by administration of a pharmaceutical composition according to the present invention.

The term “treatment” used herein refers to all actions involved in alleviating or beneficially changing symptoms of bladder cancer by administration of a pharmaceutical composition according to the present invention.

Another aspect of the present invention provides a composition for diagnosing bladder cancer, which includes an agent for measuring a methylation level of the SHH gene.

The term “diagnosis” used herein refers to confirmation of the presence or features of a pathological condition by administration of a pharmaceutical composition according to the present invention. For the purpose of the present invention, the diagnosis is to confirm the presence or absence of bladder cancer.

The inventors identified the bladder cancer prevention or treatment function of 5′-azacitidine through the inhibition of methylation of the promoter region of the Shh gene through examples.

In one embodiment of the present invention, as a result of an experiment on i) a group treated with BBN without treatment of 5′-azacitidine for 6 months and ii) a group treated with a low dose of 5′-azacitidine for 2 months from month 4 of the BBN treatment, it was confirmed that, in group i) not treated with 5′-azacitidine, invasive carcinoma was found, whereas in group ii) treated with 5′-azacitidine, invasive carcinoma was not found, indicating that the initiation of the tumor may be prevented by the 5′-azacitidine treatment before the generation of invasive carcinoma, and it was also confirmed that the anticancer initiation effect of 5′-azacitidine is mediated by the increase in stromal Hh response induced by increased Shh expression in cancer cells (see Example 3).

In another embodiment of the present invention, as a result of an experiment on i) a group not treated with 5′-azacitidine and ii) a group treated with 5′-azacitidine for 1.5 months, after orthotopic injection of BBN-induced tumor cells derived from allogeneic mice, in the control i) not treated with 5′-azacitidine, the tumor cells developed to full-fledged invasive carcinoma, whereas in group ii) treated with 5′-azacitidine, invasive carcinoma was not found, indicating that the growth of bladder tumors in immunocompetent wild-type mice was completely inhibited by the treatment of 5′-azacitidine, which is an inhibitor of DNA methylation, and it was also confirmed that the anticancer propagation effect of 5′-azacitidine is mediated by the activation of a stromal Hh signaling pathway induced by increased Shh expression in cancer cells (see Example 4).

In still another embodiment of the present invention, as a result of performing immunohistochemical analysis on BBN-induced bladder tumors in the presence of 5′-azacitidine to examine cell differentiation of transplanted tumors, it was confirmed that the expression of luminal markers increased in the tumors treated with 5′-azacitidine, and the bladder in the control exhibited increases in differentiation of squamous cells and expression of a basal subtype, and a basal phenotype, and the subtype conversion of bladder tumors is mediated by a Hh signaling pathway and Bmp (see Example 5). It was also confirmed that the subtype conversion is also observed in a human muscle-invasive urothelial carcinoma cell line or patient samples (see Example 6).

The results according to the embodiments show that 5′-azacitidine reduces a methylation level of the promoter of the SHH gene, maintains the expression level of a protein encoded by the gene to activate a normal Hh signaling pathway, thereby inhibiting the initiation or growth of bladder cancer, and converts a basal subtype to a luminal subtype of muscle-invasive bladder cancer cells to reduce tumor growth, demonstrating that 5′-azacitidine can be effectively used in the prevention or treatment of bladder cancer.

The composition for prevention or treatment according to the present invention may include a methylation inhibitor of the SHH gene as an active ingredient, and further include a pharmaceutically acceptable carrier. The pharmaceutically acceptable carrier is generally used in formulation, and includes saline, distilled water, Ringer's solution, buffered saline, cyclodextrin, a dextrose solution, a maltodextrin solution, glycerol, ethanol, liposomes, etc., but the present invention is not limited thereto. If needed, the pharmaceutically composition may further include other conventional additives including an antioxidant, a buffer, etc. In addition, by additionally adding a diluent, a dispersant, a surfactant, a binder or a lubricant, the pharmaceutical composition may be formulated as an injectable form such as an aqueous solution, an emulsion or a suspension, a pill, a capsule, a granule or a tablet. Suitable pharmaceutically acceptable carriers and their formulations may be formulated according to each ingredient using a method disclosed in the Remington's Pharmaceutical Science. The pharmaceutical composition of the present invention is not limited in dosage form, and thus may be formulated as an injection, an inhalant, or a dermal preparation for external use.

The composition for prevention or treatment of the present invention may be administered orally or parenterally (e.g., intravenously, subcutaneously, intraperitoneally, or locally), and preferably, orally, according to a desired method, and a dose of the pharmaceutical composition of the present invention may be selected according to a patient's condition and body weight, severity of a disease, a dosage form, an admistration route and duration by those of ordinary skill in the art.

The composition for prevention or treatment of the present invention is administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in the medical field. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without a side effect, and such a dose may be easily determined by one of ordinary skill in the art.

Specifically, the effective amount of the composition for prevention or treatment of the present invention may be dependent on a patient's age, sex, condition and body weight, an absorption rate of the active ingredient in the body, an inactivation rate, an excretion rate, a type of disease, or a drug used in combination, and may be generally administered at 0.001 to 150, and preferably, 0.01 to 100 mg/kg of body weight daily or every other day, or divided into one or three daily administrations. However, the effective amount may vary depending on an administration route, the severity of obesity, sex, body weight or age, and therefore, the scope of the present invention is not limited by the dose in any way.

Still another aspect of the present invention provides a method of screening a material for treating bladder cancer, which includes the following steps: (a) treating a biological sample derived from a subject with a candidate material; (b) measuring a methylation level of the SHH gene in the sample treated with the candidate material; and (c) selecting the sample as a material for treating bladder cancer when the methylation level of the SHH gene decreases, compared with a control not treated with a candidate material.

In the present invention, step (b) may include 1) treating the collected genomic DNA with a compound for modifying a non-methylated cytosine base or a methylation-sensitive restriction enzyme; and 2) amplifying the treated DNA by PCR using primers capable of amplifying a CpG island of the SHH gene promoter.

In the present invention, the compound of modifying a non-methylated cytosine base in step 1) may be bisulfite, and a method of detecting methylation of a promoter by modifying a non-methylated cytosine residue using bisulfite is widely known in the art (Herman JG et al., 1996, Proc. Natl. Acad. Sci. USA, 93: 9821-9826).

In addition, in the present invention, the methylation-sensitive restriction enzyme in step 1) is a restriction enzyme capable of specifically detecting the methylation of the CpG island as described above, and containing CG as a recognition site of the restriction enzyme. The restriction enzyme may be, for example, SmaI, SacII, EagI, HpaII, MspI, BssHII, BstUI or NotI, but the present invention is not limited thereto.

In addition, in the present invention, the amplification in step 2) may be performed by a conventional PCR method. Primers used herein are preferably designed according to the sequence of a CpG island to be targeted for analysis of methylation as described above, and may include a primer pair which can specifically amplify methylated cytosine that is not modified by bisulfite, and a primer pair which can specifically amplify non-methylated cytosine that is modified by bisulfite.

In the present invention, the subject-derived biological sample may include tissue, cells, whole blood, blood, saliva, sputum, cerebrospinal fluid or urine, and preferably, urine, but the present invention is not limited thereto.

In the present invention, the candidate material may be selected from the group consisting of a compound, a microbial culture solution or extract, a natural substance extract, a nucleic acid and a peptide, preferably, a compound, and more preferably, 5′-azacitidine, but the present invention is not limited thereto.

Yet another aspect of the present invention provides a method of preventing or treating bladder cancer, which includes administering a composition for preventing or treating bladder cancer, which includes a methylation inhibition of the SHH gene as an active ingredient, into a subject.

Yet another aspect of the present invention provides a use of the composition for preventing or treating bladder cancer.

Hereinafter, to help in understanding the present invention, exemplary examples will be suggested. However, the following examples are merely provided to more easily understand the present invention, and not to limit the present invention.

### EXAMPLES

### Example 1

### Experimental Preparation and Methods

1-1. Mice

For a gene deletion experiment, Col1a2^{CreER} (RRID: IMSR_JAX: 029235) mice were mated with the Smo^{flox/flox} (RRID: IMSR_JAX: 007926) or Gli2^{flox/flox} (RRID: IMSR_JAX: 004526) strains, thereby obtaining Col1a2^{CreER}; Smo^{flox/flox} or Col1a2^{CreER};Gli2^{flox/flox} mice.

The mice were administered 8 mg of tamoxifen (TM; Sigma) per 30 g of body weight by oral gavage for three consecutive days. Male mice aged 8 to 10 weeks were used. For experiments associated with 5′-azacitidine (Sigma), 1 mg of 5′-azacitidine per kg of body weight was intraperitoneally injected into the mice daily. The dosing duration is described in the brief description of the drawings. In each experiment, the mice were randomly selected for a drug/TM or control-treated group. The experiments involving mice were performed under isoflurane anesthesia. All procedures were performed according to a protocol approved by the Institutional Animal Care and Use Committee at POSTECH (IACUC number: POSTECH-2017-0094).

1-2. BBN-Induced Bladder Carcinogenesis

0.1% N-butyl-N-4-hydroxybutyl nitrosamine (BBN, TCI) was dissolved in drinking water, and the BBN-containing water was placed in a dark bottle, and provided ad libitum to mice for 4 to 6 months. The BBN-containing water was replaced twice a week. Bladders were collected and analyzed 4 to 6 months after BBN administration.

1-3. Analysis of Genomic DNA Methylation Using Bisulfite Sequencing

The DNA methylation status of mouse and human Shh was confirmed using genomic DNA bisulfite sequencing. For bisulfite conversion, 1 μg of genomic DNA was converted using a MethylEdge Bisulfite Conversion System (Promega) according to the manufacturer's instructions. The genomic sequences of the regulatory regions of mouse Shh and human SHH were obtained from the NCBI nucleotide database (Mus musculus: NC_000071.6, Homo sapiens: NG_007504.2), and the CpG island (island) and CpG shores in the regulatory region were identified using Methprimer 2.0 (Li and Dahiya, 2002) (RRID: SCR_010269). The 2kb regions upstream and downstream of the CpG island were referred to as a “CpG upshore” and a “CpG downshore,” respectively. For sequencing analysis, bisulfite-converted DNA was amplified by EpiTaq HS (TaKaRa), and the CpG island and CpG shore regions were subcloned into a pGEM-T easy vector (Promega). The region containing the CpG island and CpG shore was divided into 8 sub-regions, and each sub-region was amplified using specific primers designed for bisulfite-converted target sequences. The primers used for amplification are shown in Table 1 below.

TABLE 1

| Target | Primer | | |
|---|---|---|---|
| species | name | Forward sequence (5′-3′) | Reverse sequence (5′-3′) |
| Mouse | Shh | TTTTTAGTTTTGTTATTATTTAAAATT | CAAAAATCACCAAAAAACATCTAAC |
| | promotor | AGG | |
| | Shh upshore | TTTGTATATTTATATTTGGGGATGG | AAAAAACTTATAAAACAAACTACCTTT |
| | region 1 | | C |
| | Shh upshore | TTGTATTTTGTTAGGATAGATTGGAAG | ACCCCATCCCCAAATATAAATATAC |
| | region 2 | | |
| | Shh upshore | GGATGGTGAGGTTTTGTTATATTGT | GGATGGTGAGGTTTTGTTATATTGT |
| | region 3 | | |
| | Shh upshore | TGAAGTAAAATGAGGTTTTAGGATGT | CACCATCCCAAACTTAAAAAAATTA |
| | region 4 | | |
| | Shh | ATGTTGTTGTTGTTGGTTAGATGTT | ATAAAAAACCCCATCTTCTAATACC |
| | downshore | | |
| | region 1 | | |
| | Shh | GGGTATTAGAAGATGGGGTTTTTTA | CCCAAACTTTCTCAATTACAATTCT |
| | downshore | | |
| | region 2 | | |
| | Shh | GAAAGTTTGGGGGTAGTTTTGATA | TATTTACAAAAAAACCCATTTCCAA |
| | downshore | | |
| | region 3 | | |
| Human | Shh | TTTTTTTGTTTTTTGATTGTTGTTT | TCAACTTTTTAAAATACCTCCTCTTC |
| | promotor | | |
| | Shh upshore | TTTTGGGGAAGAAAAATTAAATAAT | CAACAATCAAAAAACAAAAAAAATCT |
| | region 1 | | A |
| | Shh upshore | AGTGAGGTGATTATAGATTTAAAGAT | CAACTATTATTTAATTTTTCTTCCCC |
| | region 2 | | |
| | Shh upshore | ATTTGTAAAGGGAATTTTTGGAAAT | AACCAAAAAAATAAAATTTAAAACTCC |
| | region 3 | | |
| | Shh upshore | TGTTAAGGGTGGAAGGTAGGGTAGT | CAAAAATTCCCTTTACAAATCAACT |
| | region 4 | | |
| | Shh | GGAAGAGGAGGTATTTTAAAAAGTTG | AACTAAACCCTTAACCTCCATTCTC |
| | downshore | | |
| | region 1 | | |
| | Shh | GAGAATGGAGGTTAAGGGTTTAGTT | CCTCCTAACTTTTCCAATTAAAAA |
| | downshore | | |
| | region 2 | | |
| | Shh | ATTTTTAATTGGAAAAGTTAGGAGG | CAAAAAAACCCATTTCTAACTTCAA |
| | downshore | | |
| | region 3 | | |

The sequencing data was assembled using SnapGene software (https://snapgene.com/, RRID: SCR_015053) and the MUSCLE: multiple sequence alignment tool (haps://www.ebi.ac.uk/Tools/msa/muscle/ RRID: SCR_011812). The average degree of methylation was obtained from the analysis of 8 to 10 clones of each sub-region. The methylated CpG sites were counted and distinguished from unmethylated CpG sites.

1-4. Bladder Organoid Cell Culture

BBN-induced bladder tumors were minced, and then incubated in DMEM (Gibco) containing collagenase I and II (20 mg/ml each) and thermolysin (250 KU/ml) at 37° C. for 2 hours, followed by 5-minute trituration every 30 minutes. A single cell suspension was obtained and filtered through a 100 μm cell strainer (Falcon). After lysis of red blood cells in ACK lysis buffer (Gibco), the cells were washed with DMEM containing 10% fetal bovine serum (Millipore) and counted using a hemocytometer (Sigma).

For bladder organoid culture, single tumor cells were overlaid on growth factor-reduced Matrigel (Corning), and incubated in advanced DMEM/F-12 (Gibco) supplemented with 10 mM HEPES (pH 7.4, Sigma), 10 mM Nicotinamide (Sigma), 1 mM N-acetyl-L-cysteine (Sigma), GlutaMAX (Gibco), 1% penicillin/streptomycin (Gibco), 50 ng/ml mouse EGF (Peprotech), 0.5X B-27 (Gibco), 1 mM A8301 and 10 mM Y-27632.

For Bmp4 treatment, organoids were treated with a recombinant Bmp4 protein (Peprotech) for 8 days, and the medium was changed every two days.

For knock-down experiments, bladder tumor organoids were infected with a lentivirus containing shRNA specific for mouse/human Shh or Bmpr1a (Minis Bio).

TABLE 2

| Target | | | | |
|---|---|---|---|---|
| species | shRNA | Target sequence | Sense | Antisense |
| Mouse | Bmpr1a | CTTTAGCCTACAAGCA | GGGUCGUUACAACCGU | AAAUCACGGUUGUAAC |
| | | GTTTA | GAUUU | GACCC |
| | Shh | CTTTAGCCTACAAGCA | CUUUAGCCUACAAGCAG | UAAACUGCUUGUAGGC |
| | | GTTTA | UUUA | UAAAG |
| Human | Bmpr1a | GTCCAGATGATGCTATT | GUCCAGAUGAUGCUAU | UAUUAAUAGCAUCAUC |
| | | AATA | UAAUA | UGGAC |
| | Shh | CTACGAGTCCAAGGCA | CUACGAGUCCAAGGCAC | AUAUGUGCCUUGGACU |
| | | CATAT | AUAU | CGUAG |

A collected supernatant was filtered through a 0.45-μm pore PES filter (Millipore) 48 hours after transfection. A viral titer was calculated from 3T3 cells by serial dilution of the virus-containing supernatant. For lentivirus infection, bladder organoids were incubated in a lentivirus-containing medium with polybrene (8 mg/ml, Sigma) for 12 hours at 37° C. Infected organoids, which were GFP- or mCherry-positive, were selected using a fluorescence microscope.

1-5. Orthotopic Transplantation of Bladder Tumors

Bladder tumors were dissociated into single cells as described above. The cells were resuspended in 80 ml DMEM containing 50% Matrigel (BD Bioscience), and then submucosally injected into the anterior aspect of the bladder dome using a 29-gauge insulin syringe. An abdominal incision and skin were closed with a 4-0 nylon suture, and the surgical site was disinfected with alcohol. Bladder tumor organoids were selected, and resuspended in a 50% organoid medium and 50%

Matrigel, followed by transplantation into recipient mice.

1-6. Human Bladder Tumor Samples and Cancer Cell Lines

Frozen human bladder tissue samples were obtained from the tissue bank of Seoul National University Hospital. For fresh bladder tumor samples, 0.5 to 1-cm³ bladder tissue specimens were obtained from patients undergoing cystectomy or TURBT according to a protocol approved by the SNUH Institutional Review Board (IRB No.: 1607-135-777). Informed consent to patient information provision and publishing was obtained from the patients. The cancer tissues were evaluated before transport to POSTECH for additional analysis. For experiments for bladder cancer cell lines, J82 (RRID: CVCL_0359), T24 (RRID: CVCL_0554) and TCC (RRID: CVCL_1738) were used. All cell lines were authenticated by a STR profiling method, and tested negative for mycoplasma contamination.

1-7. Quantitative RT-PCR

Human or mouse bladder samples were snap-frozen in liquid nitrogen, homogenized with a mortar and a pestle, and RNA was extracted using an RNeasy Plus Mini Kit (Qiagen).

Subsequently, the RNA samples were dissolved in RNase-free water, and their concentration and purity were measured with a spectrophotometer. The TAE/formamide electrophoresis method (Masek et al., 2005) was used for RNA quality analysis. For quantitative RT-PCR of mRNA transcripts, first-strand cDNA was synthesized using a high-capacity cDNA reverse transcriptase kit (Applied Biosystems) containing oligo dT. Quantitative RT-PCR was performed using SYBR Green Supermix (Applied Biosystems) and a one-step cycler (Applied Biosystems), and gene expression was normalized to the housekeeping gene HPRT1.

1-8. Histological Analysis

Tumor specimens were fixed in 10% neutral-buffered formalin for 12 hours, embedded in paraffin, and then sectioned into 4-um thick sections using a microtome. The slides were stained with hematoxylin and counter-stained with eosin for histological analysis. For immunostaining, tumor samples were embedded in an OCT compound (Tissue-Tek) and sectioned into 10-um-thick sections with a cryostat (Leica).

1-9. Immunofluorescence Analysis of Tissue Sections

Bladder tumors separated from mice were fixed in 10% neutral-buffered formalin for 3 hours, washed with PBS three times, incubated in 30% sucrose overnight, and embedded in an OCT compound (Tissue-Tek).

Subsequently, the sections prepared by the above procedure were washed in PBS twice, blocked in 2% goat serum containing 3% BSA in PBS containing 0.25% Triton X-100 for 1 hour, and incubated overnight at 4° C. in a humidified chamber with primary antibodies diluted with a blocking solution.

Afterward, the sections were washed with PBS containing 0.25% Triton X-100 three times, and incubated with suitable Alexa Fluor-conjugated secondary antibodies diluted in 1:1000 with a blocking solution at room temperature for 1 hour.

Finally, the sections were washed with PBS three times, and tissue sections were mounted with a Prolong Gold mounting reagent (Invitrogen). All immunofluorescence images were analyzed by confocal microscopy (Leica SP5 or Olympus FV1000).

1-10. Construction of RNA-Seq Libraries

Total RNA was extracted with a TRIzol reagent (Thermo Fisher) according to the manufacturer's instructions. RNA-seq libraries were constructed using the TruSeq sample prep kit V2 (Illumina). An amount of the RNA-seq library was determined by Nanodrop, and the average amount of the RNA-seq libraries ranged from 30 to 50 ng/ml. The RNA-seq libraries were sequenced using a NextSeq platform with 75-bp single-end reads.

1-11. Differential Gene Expression and Gene Set Enrichment Analysis (GSEA) of RNA-Seq Data

Differentially expressed genes were analyzed using Cufflinks tools (Trapnell et al., 2012). From all annotated genes, genes were removed when the rpkm average of all sequenced samples is less than 1.0, such that the depth to which the genes are assigned may be low. GSEA was performed according to the instructions (RRID: SCR_003199). To generate a customized gene set for a luminal marker and a basal marker, a representative gene for each signature was obtained from a previous study (Damrauer et al., 2014). The RNA-seq data set used herein was deposited in NCBI GEO (Accession No.: GSE129441).

1-12. Data Analysis

Statistical analysis was performed using GraphPad Prism software v.6 (RRID: SCR_015807). All data was represented as the mean±SEM, and two groups were compared using a two-tailed Student's test. P<0.05 was considered statistically significant. For TCGA data analysis, gene expression levels of muscle-invasive bladder cancer patients were downloaded from the TCGA data portal (https://portal.gdc.cancer.gov/).

A FPKM expression value was log2 (x+1) transformed for convenient comparison of mRNA abundance estimates, where x denotes the FPKM value for each gene. The log-transformed expression value was normalized to a z-score for additional analysis. Gene Cluster 3.0 was used for unsupervised hierarchical clustering (de Hoon et al., 2004), and as default settings, similarity metric and clustering methods for uncentered correlation and centroid linkage were set, respectively. Visualization of the mRNA cluster results was performed using Java TreeView (Saldanha, 2004) (RRID: SCR_016916). To examine the clinical results of different mRNA clusters, survival analysis was conducted using an Oasis2 tool (Han et al., 2016). In a Kaplan-Meier survival test, patients with a survival rate of 5 years or less were considered for survival analysis. The Oncoprint format of mutagenesis was plotted using cBioPortal (Cerami et al., 2012, Gao et al., 2013) (RRID: SCR_014555).

### Example 2

### Confirmation of Correlation Between Methylation of Shh Promoter Region and Shh Expression in Muscle-Invasive Urothelial Carcinoma

2-1. Confirmation of Role of 5′-Azacitidine in Methylation of Shh Promoter Region and Shh Expression in Mice with Muscle-Invasive Urothelial Carcinoma

To confirm the role of 5′-azacitidine in methylation of the Shh promoter region and Shh expression in a mouse with muscle-invasive urothelial carcinoma, an animal obtained one week after orthotopic transplantation of a BBN-induced mouse tumor was treated with 5′-azacitidine (1 mg per kg of body weight of mouse) every other day for 2 weeks before methylation analysis, followed by bisulfite sequencing analysis (unpaired Student's t test (**, p<0.001). n=3, the entire experiment was repeated three times).

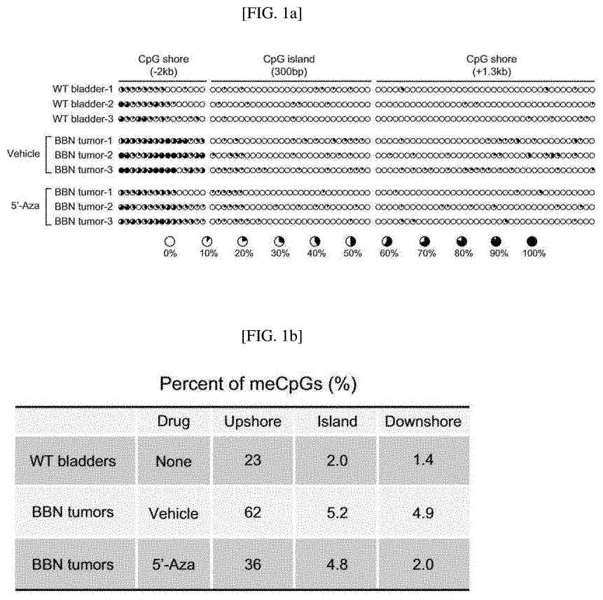

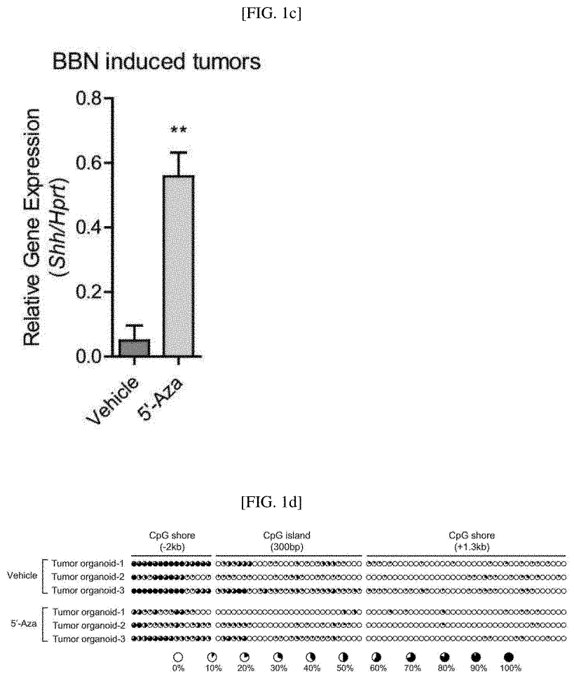

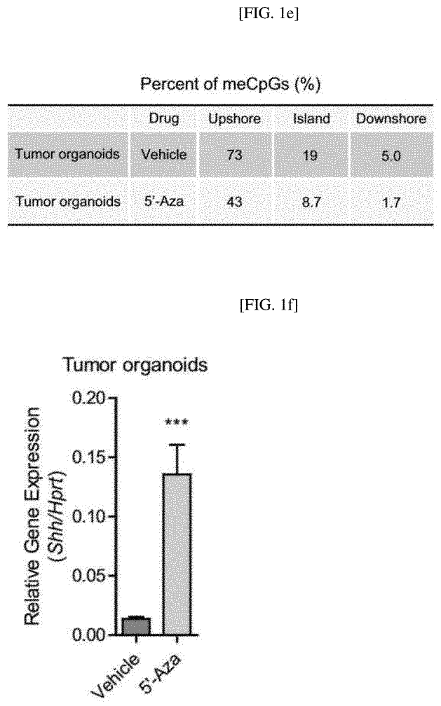

As a result, as shown in FIGS. 1A and 1B, a methylation level in the CpG shore upstream of the CpG island of the Shh promoter region was detected at 62% in a vehicle control, but 36% in a 5′-azacitidine-treated experimental group.

In addition, as shown in FIG. 1C, by the orthotopic transplantation of a BBN-induced mouse tumor and 5′-azacitidine treatment, it was confirmed that a Shh gene expression level 11-fold increased compared to the vehicle control.

2-2. Confirmation of Role of 5′-Azacitidine in Methylation and Shh Expression of 3D Tumor Organoids

3D bladder tumor organoids were obtained by orthotopically transplanting bladder tumors induced by BBN, in addition to primary tumors of mice. The histopathological characteristics of parental tumors may be identified from the organoids, and the pathological characteristics of BBN-induced urothelial carcinoma were able to be reproduced. Tumor organoids were cultured using a Matrigel overlay method, and three days after seeding, the tumor organoids were treated with 5′-azacitidine (1 μM) for four consecutive days, followed by bisulfite sequencing analysis (unpaired Student's t test (**, p<0.001). n=3, the entire experiment was repeated three times).

As a result, as shown in FIGS. 1D and 1E, a methylation level in the CpG shore upstream of the CpG island of the Shh promoter region was detected at 73% in a vehicle control, but 43% in a 5′-azacitidine-treated experimental group.

In addition, as shown in FIG. 1F, by 5′-azacitidine treatment, it was confirmed that a Shh gene expression level 9-fold increased compared to the vehicle control.

The above results are consistent with those in muscle-invasive urothelial carcinoma-induced mice (Example 2-1).

### Example 3

### Confirmation of Inhibition of Urothelial Carcinoma Initiation by 5′-Azacitidine and its Mechanism

3-1. Confirmation of Inhibition of Urothelial Carcinoma Initiation by 5′-Azacitidine

From the result of confirming that Shh expression reduced in mice with urothelial carcinoma and bladder tumor organoids is recovered after 5′-azacitidine treatment (see Example 2), it was deduced that the inhibition of DNA methylation would suppress the development of bladder cancer at the early stage of tumor initiation.

To verify the deduction, an experiment for inhibiting DNA methylation using 5′-azacitidine in a BBN-induced bladder cancer model was performed.

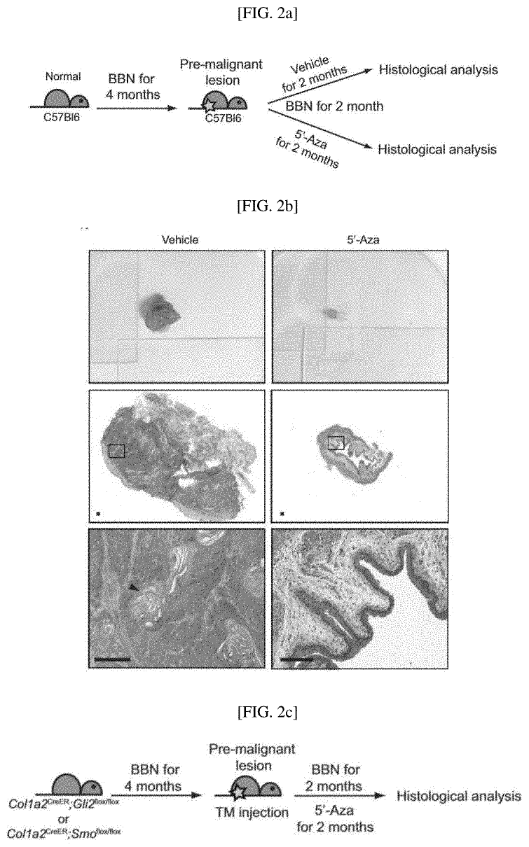

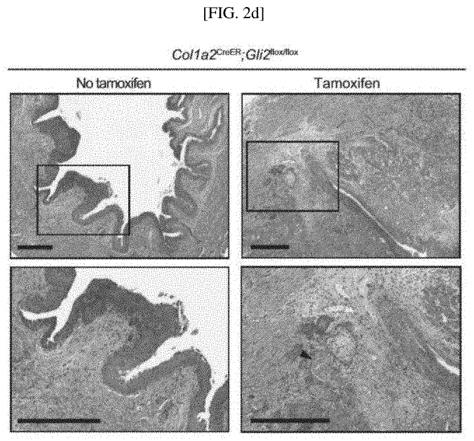

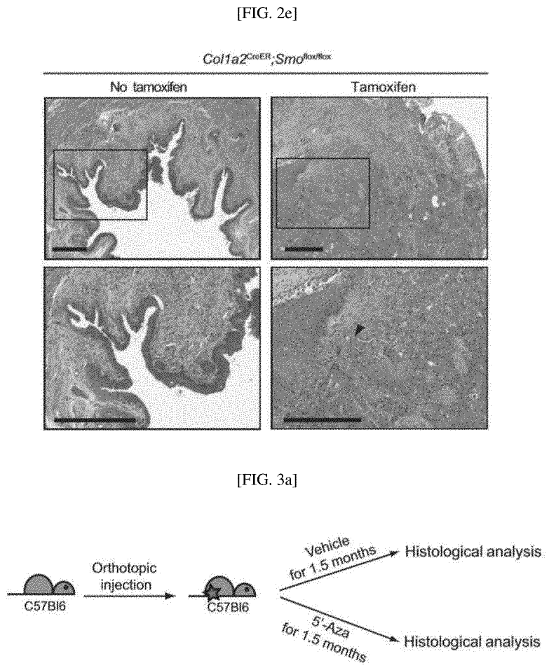

More specifically, to induce carcinoma in situ (CIS) lesions, mice (14 animals) exposed to BBN for 4 months were divided into a vehicle control (7 animals) and a 5′-azacitidine-treated group (7 animals), and each group was treated with a vehicle or 5′-azacitidine for 2 months and continued exposure to BBN, thereby inducing invasive carcinoma before histopathological analysis of bladder. The experimental scheme is shown in FIG. 2A, and bladder sections of mice treated with the vehicle control and 5′-azacitidine were subjected to H&E staining

As a result, as shown in FIG. 2B, invasive carcinoma was found in the vehicle control, but not found in the 5′-azacitidine-treated group.

The above results showed that, when DNA methylation is inhibited before generation of invasive carcinoma, tumor initiation is suppressed.

3-2. Confirmation of Mechanism of Anticancer Initiation by 5′-Azacitidine

It is reported that the loss of a stromal Hedgehog response causes the initiation of muscle-invasive urothelial carcinoma, the increase in Hh signaling inhibits the development of bladder cancer at the early stage of progression, Shh expression is exhibited in basal stem cells of the urothelial epithelium, and the response to this signal is limited by the stroma.

Therefore, to confirm whether the anticancer initiation effect of 5′-azacitidine is mediated by the increase in a stromal Hh signaling response, which is caused by increased Shh expression in cancer cells, an experiment for confirming whether the tumor suppressing effect of 5′-azacitidine is still observed when a Hh response in the stroma is genetically inhibited.

More specifically, to genetically inhibit the stromal Hh response, a Col1a2^{CreER}; Smo^{flox/flox} strain (10 mice) or Col1a2^{CreER}; Gli2^{flox/flox} strain (10 mice), which expresses tamoxifen (TM)-inducible stroma-specific CreER (Col1a2^{CreER}) and carrying homozygous floxed alleles (Gli2 or Smoothened) that are essential factors of the Hh pathway were used. In addition, the mice were exposed to BBN for 4 months, and then injected with TM (5 mice per strain, genetic removal of stromal Hh response before generation of muscle-invasive carcinoma) or corn oil (5 mice per strain) for three consecutive days, and then the mice were further exposed to BBN for 2 months in the presence of 5′-azacitidine. The experimental scheme is shown in FIG. 2C, and bladder sections of mice treated with the vehicle control and 5′-azacitidine were subjected to H&E staining.

As a result, as shown in FIGS. 2D and 2E, to remove the Hh response from the stroma, it was confirmed that the anticancer initiation effect of 5′-azacitidine was reversed in the TM-treated group, and muscle-invasive urothelial carcinoma appeared at 6 months of the exposure, which is the same as that in the BBN-exposed normal mice, whereas no muscle-invasive urothelial carcinoma was observed in the vehicle control.

This result showed that the DNA methylation of the Shh gene serves as the molecular basis for losing Shh expression in muscle-invasive urothelial carcinoma, and the stromal Hh signal plays a critical role in the initiation of bladder cancer at the early stage.

### Example 4

### Confirmation of Inhibition of Growth of Urothelial Carcinoma by 5′-Azacitidine and its Mechanism

4-1. Confirmation of Inhibition of Growth of Urothelial Carcinoma by 5′-Azacitidine

It was confirmed that the activation of a stromal Hh signaling pathway induced by inhibiting DNA methylation inhibits the metastasis of pre-cancerous lesions to muscle-invasive cancer at the early stage of tumorigenesis. However, it is not certain whether the inhibition of DNA methylation exhibits an effect of inhibiting the growth of mature urothelial carcinoma.

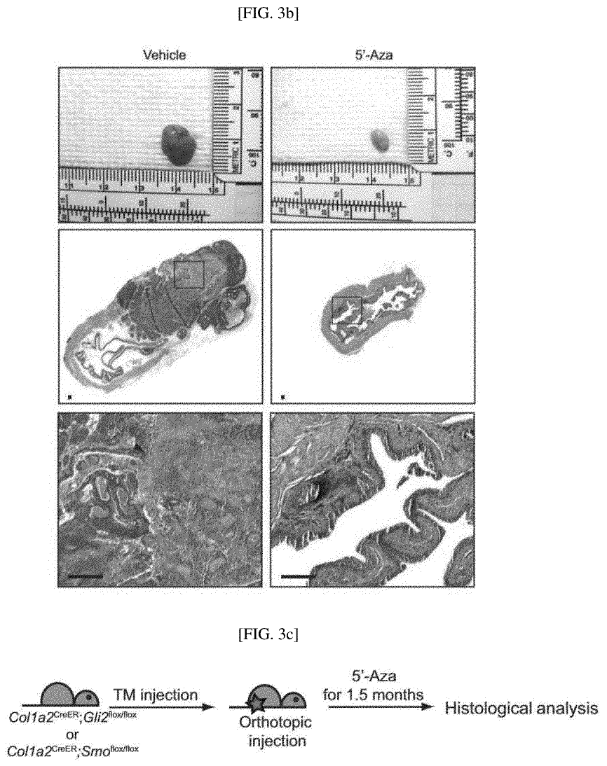

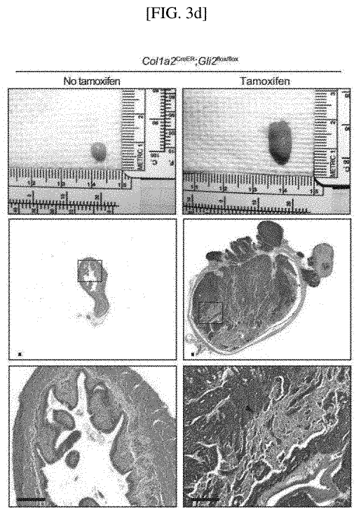

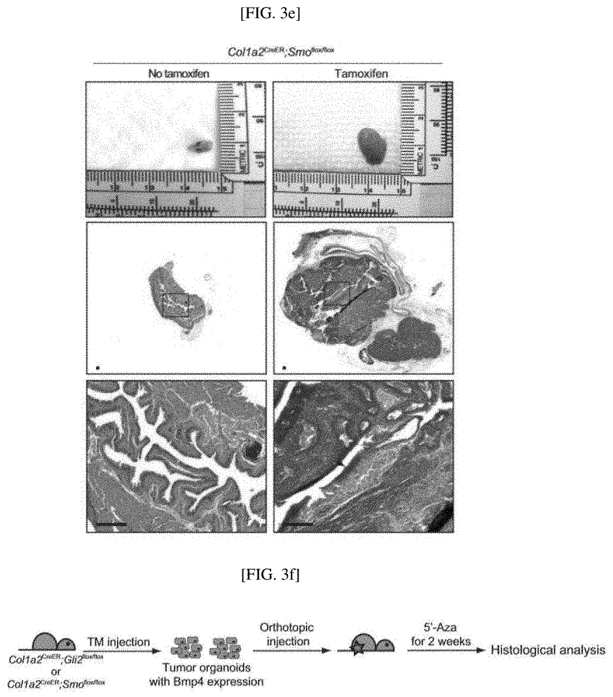

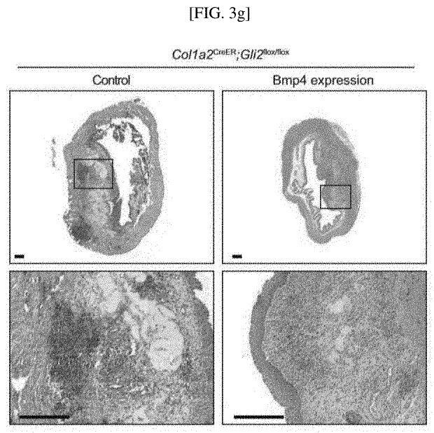

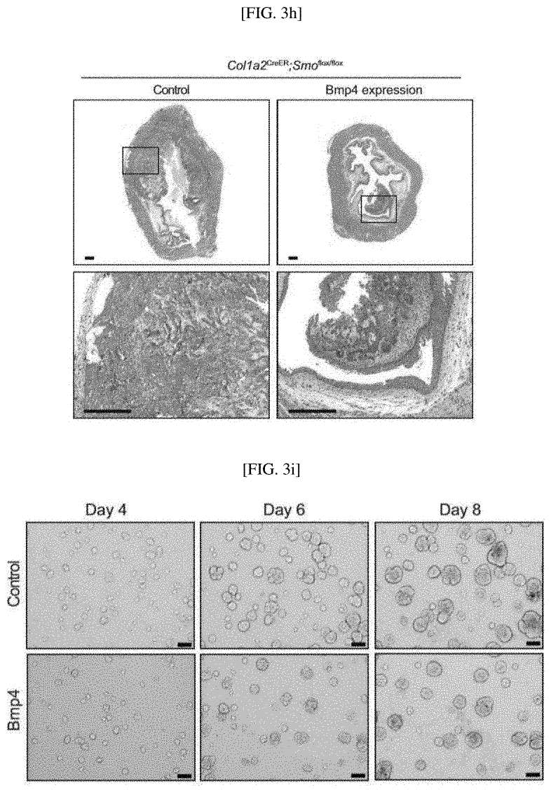

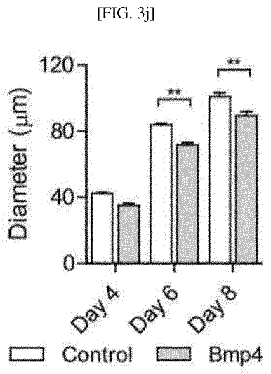

To evaluate the effect of the inhibition of DNA methyltransferase (DNMT) on the growth of bladder cancer, recently established transplantation models were used, and these models allow the proliferation of tumor cells transplanted in a microenvironment in vivo by injecting bladder cancer cells into the wall of the bladder dome. Mice orthotopically injected with BBN-induced bladder tumor cells (14 mice) were divided into a vehicle control (7 mice) and a 5′-azacitidine-treated group (7 mice), and then treated with a vehicle and 5′-azacitidine for 1.5 months, respectively. The experimental scheme is shown in FIG. 3A, and sections in the vehicle control or the 5′-azacitidine-treated group were subjected to H&E staining.

As a result, as shown in FIG. 3B, it was confirmed that invasive carcinoma appeared in the vehicle control, whereas no invasive carcinoma appeared in the 5′-azacitidine-treated group.

These results showed that the inhibition of the DNA methylation by 5′-azacitidine completely inhibits the growth of bladder tumors in immunocompetent wild-type mice.

4-2. Confirmation of Mechanism of Anticancer Propagation Effect by 5′-Azacitidine

To confirm whether the anticancer propagation effect of 5′-azacitidine is mediated by the activation of the stromal Hh signaling pathway induced by increased Shh expression in cancer cells, an experiment was performed by a combination of a pharmacological approach to 5′-azacitidine treatment for increasing the Shh expression in tumors and a genetic approach to genetically inhibit the stromal Hh signaling pathway.

More specifically, to genetically inhibit the stromal Hh signaling pathway in mice, Col1a2^{CreER}; Gli2^{flox/flox} and Col1a2^{CreER}; Smo^{flox/flox} strains were used, and after TM injection for three consecutive days, BBN-induced tumors derived from allogeneic mice were orthotopically transplanted, and then 5′-azacitidine was treated for 1.5 months. The experimental scheme is shown in FIG. 3C, and sections of the vehicle control or TM-treated group were subjected to H&E staining.

As a result, as shown in FIGS. 3D and 3E, it was confirmed that the anticancer propagation effect of 5′-azacitidine disappeared in the strains in which the stromal Hh signaling pathway is genetically inhibited.

These results showed that the tumor cell proliferation inhibitory effect of 5′-azacitidine is mediated by a Shh-induced stromal Hh signaling pathway, and the Shh expression is epigenetically regulated by cancer cells.

4-3. Confirmation of Anticancer Propagation Effect by 5′-Azacitidine

An experiment was performed to confirm whether the Hh signaling-mediated anticancer propagation effect is regulated by Bmp. Here, the Bmp is a secreted stromal factor known to be regulated by the stromal Hh signaling pathway in the bladder. Bmp is secreted stromal factor involved in urothelial differentiation, and it is reported that the activation of the Bmp pathway hinders bladder cancer progression prior to the generation of muscle-invasive carcinoma by stimulating urothelial differentiation. However, the role of stromal Bmp in the late stage of tumor development, particularly, tumor growth, is not known.

To confirm whether the Bmp expression regulated by the stromal Hh signaling pathway affects bladder cancer growth, an experiment of overexpressing Bmp4 in bladder tumor organoids derived from BBN-induced tumors was performed, and the Bmp4 expression in the organoids 10-fold increased compared with the control organoids. The Bmp4-expressing organoids were orthotopically injected into Col1a2^{CreER}; Smo^{flox/flox} (8 mice) and Col1a2^{CreER}; Gli2^{flox/flox} (8 mice) mice, and injected with TM for three consecutive days. Subsequently, the Bmp4-expressing bladder tumor organoids were orthotopically injected into the mice, and then treated with 5′-azacitidine for 2 weeks. The experimental scheme is shown in FIG. 3F, and a wild-type bladder tumor organoid-orthotopically injected control and the Bmp4-expressing tumor organoids were strained by H&E staining.

Results obtained through H&E staining of sections of the wild-type bladder tumor organoid-orthotopically injected control and the Bmp4-expressing tumor organoids are shown in FIGS. 3G and 3H.

In addition, tumor organoids derived from BBN-induced bladder tumors were cultured in the absence or presence of Bmp4 for 8 days.

As a result, as shown in FIG. 3I, bright-field images of the cultured tumor organoids were confirmed.

In addition, as shown in FIG. 3J, the average size of the bladder tumor organoids cultured for 4, 6 and 8 days in the absence or presence of the Bmp4 protein was confirmed (n=90 in each condition).

In addition, as shown in FIG. 3K, quantification results for cell proliferation in the tumor organoids cultured for 6 days in the absence or presence of the Bmp4 protein were confirmed. Images immunostained with DAPI and Ki67 were confirmed, and represented as percentages of DAPI-stained nuclei (unpaired Student's t test (**, p<0.01)).

The results show that, while the methylation of the Shh promoter region is suppressed by 5′-azacitidine treatment in in vivo tumor cells to express Shh at a normal level, the tumor inhibitory effect of Bmp was confirmed when the stromal Hh signaling pathway was inhibited using Col1a2^{CreER}; Smo^{flox/flox} or Col1a2^{CreER}; Gli2^{flox/flox} mice, which demonstrates that the Shh expression induced by decreased methylation in cancer cells activates the Hh signaling pathway in bladder stroma, thereby increasing stromal expression of Bmp, which sends a signal back to the tumor cells, and inhibiting the growth of cells, and supports a potential scenario of an increased reciprocal tumor-stromal signal feedback loop.

### Example 5

### Confirmation of Effect of 5′-Azacitidine on Urothelial Carcinoma Subtype Differentiation

5-1. Confirmation of Inhibition of Growth of Urothelial Carcinoma by 5′-Azacitidine

To investigate the cellular basis of the cancer suppressive effect of the Shh-induced stromal Hh signaling pathway regulated by DNA methylation of 5′-azacitidine in tumor cells, BBN-induced tumors were orthotopically injected into nude mice.

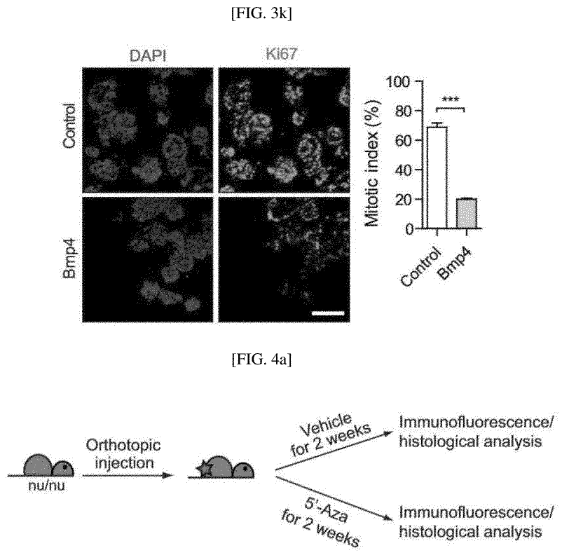

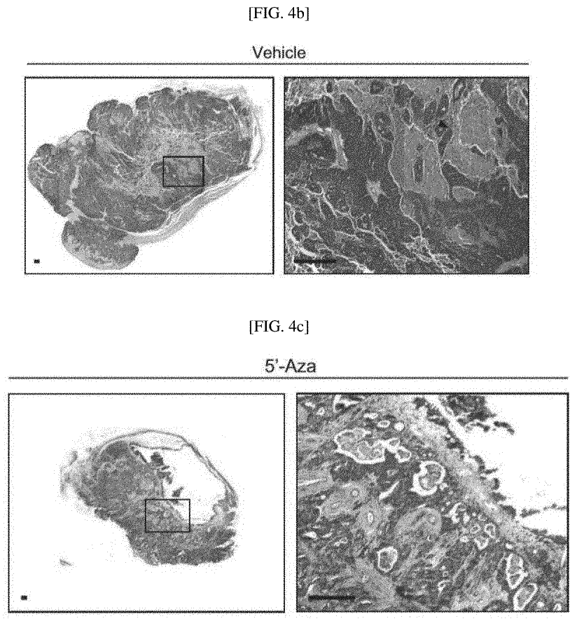

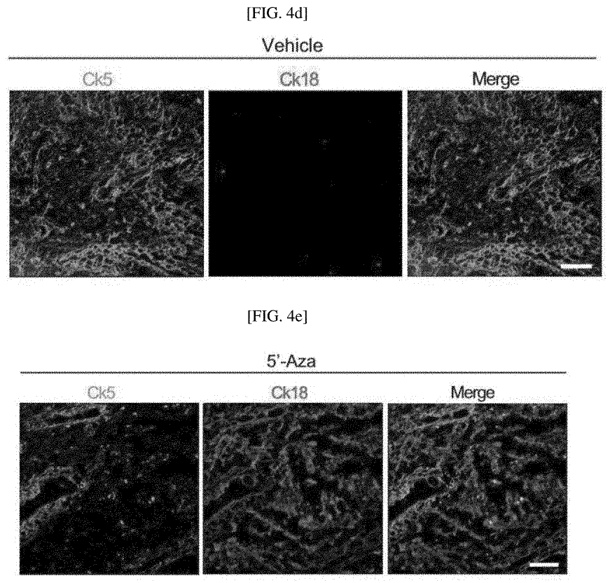

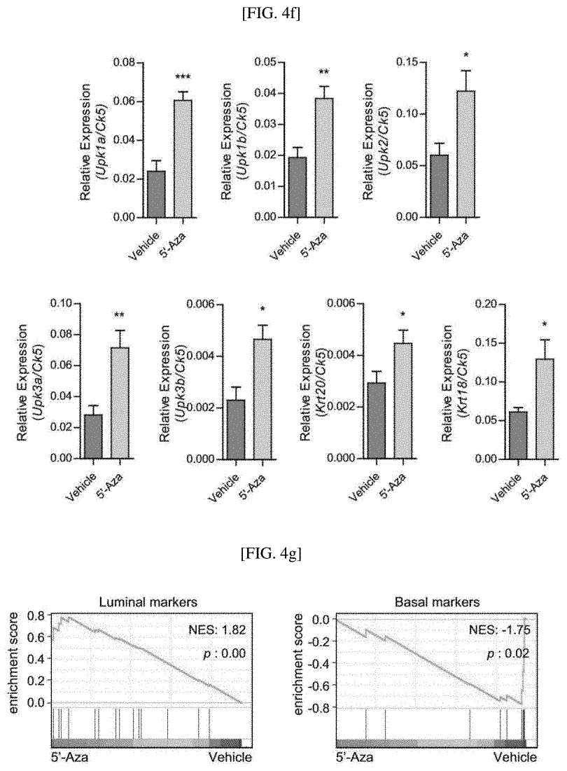

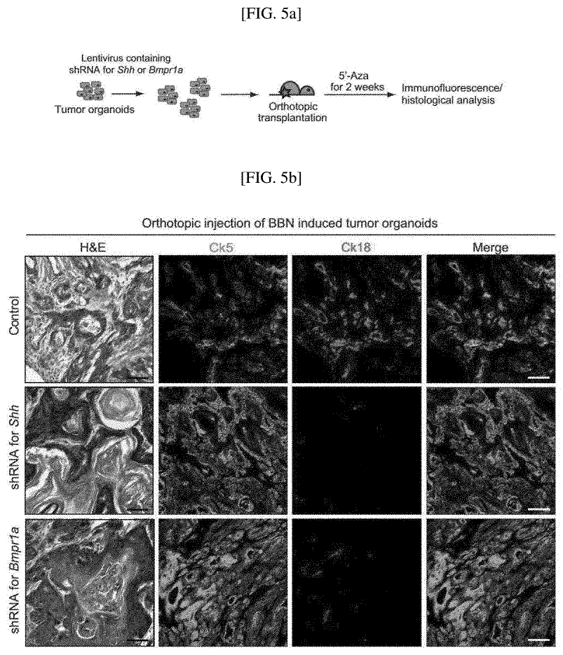

When the bladder tumors are orthotopically transplanted into wild-type mice in the presence of 5′-azacitidine, tumor growth is completely blocked. Therefore, the nude mice were selected to grow the transplanted tumors under more mild conditions, and facilitate research on the basis of the anticancer effect of the stromal Hh signaling pathway induced by the suppression of Shh methylation on tumor growth. To evaluate the effect of DNA methyltransferase inhibition on the growth of bladder cancer under immunocompromised conditions, nude mice (14 mice) orthotopically injected with BBN-induced bladder tumor cells were divided into a vehicle control (7 mice) and a 5′-azacitidine-treated group (7 mice), and then treated with a vehicle and 5′-azacitidine for 2 weeks, respectively. In addition, allograft sections of the vehicle control or 5′-azacitidine-treated mice were subjected to H&E staining, and the experimental scheme is shown in FIG. 4A.

As a result, as shown in FIGS. 4B and 4C, H&E staining images of the allografts of the vehicle control or 5′-azacitidine-treated group were confirmed. More specifically, the bladder tumors transplanted into the nude mice grew but have smaller tumor lesions under the 5′-azacitidine treatment, as compared with a group not treated with 5′-azacitidine.

These results show that the 5′-azacitidine treatment is still effective in inhibiting tumor growth under immunocompromised condition, which is consistent with the above-described result in that the inhibition of the DNA methylation completely inhibits bladder tumor growth in immunocompetent wild-type mice (see Example 4).

5-2. Confirmation of Subtype Conversion by 5′-Azacitidine

As described above, it has been reported that the anticancer effect of the Hh signal is mediated by stromal Bmp, and the Bmp signaling activity is associated with differentiation of basal cells to luminal cells, and in the research on the cellular origin of bladder cancer, it has been reported that urothelial carcinoma is derived from basal stem cells. In addition, based on the expression level of basal markers and a mutational profile, the muscle-invasive carcinomas generated in the BBN models are reported to be most similar to the basal subtype of human urothelial carcinoma, which is the most aggressive form of bladder cancer.

Therefore, it was assumed that the increased activity of a Hh signaling pathway might make tumors differentiate into the less aggressive luminal subtype. The luminal subtype of tumor exhibits very slow growth upon 5′-azacitidine treatment.

To investigate the cell differentiation of transplanted tumors, tumor allografts of the vehicle control or 5′-azacitidine-injected mice were immunostained. The immunostaining results showed, as shown in FIGS. 4D and 4E, that the expression of a luminal subtype marker, Ck18, was increased in the tumor allografts of the 5′-azacitidine-injected mice, and a basal phenotype and the expression of a basal subtype marker, Ck5, were shown in the control (the basal subtype marker, Ck5, was represented in green, and the luminal subtype marker, Ck18, was represented in red).

In addition, according to the quantitative RT-PCR experiment, as shown in FIG. 4F, it was confirmed that the expression of the luminal marker was increased, and more particularly, the expression of the luminal markers such as Upk1a, Upk1b, Upk2, Upk3a, Upk3b, Krt20 and Krt18 increased 3-fold, 2-fold, 2-fold, 2.5-fold, 2-fold, 1.5-fold and 2-fold, respectively, in the tumor allografts of the 5′-azacitidine-injected mice, compared with the vehicle control (the gene expression was normalized to a basal marker Krt5, an unpaired Student's t test (*, p<0.05; **, p<0.01; ***, p<0.001; n=3; the entire experiment was repeated 6 times).

In addition, using standard luminal and basal signatures obtained from previous research, GSEA of tumor allografts treated with the vehicle control and 5′-azacitidine from RNA-Seq data was performed.

As a result, as shown in FIG. 4G, it was confirmed that tumors growing in the presence of 5′-azacitidinee expressed basal markers at a relatively low level, and exhibited a strong luminal signature. However, it was confirmed that the vehicle control, allografts, growing in the absence of 5′-azacitidine shows clear standard signature of a basal subtype.

These results show that the activation of the Hh signaling pathway, induced by epigenetically upregulated Shh expression in tumor cells induces the conversion of bladder cancer cells from a basal subtype to a luminal subtype, which can explain the reduced tumor growth.

5-3. Confirmation of Association of Hh Signaling Pathway with Subtype Conversion of Bladder Cancer

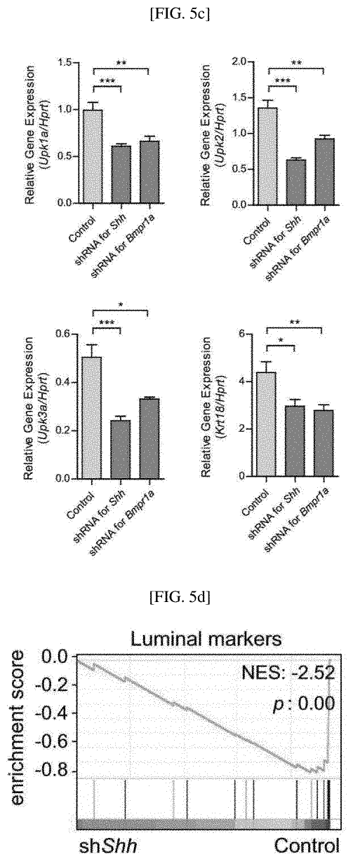

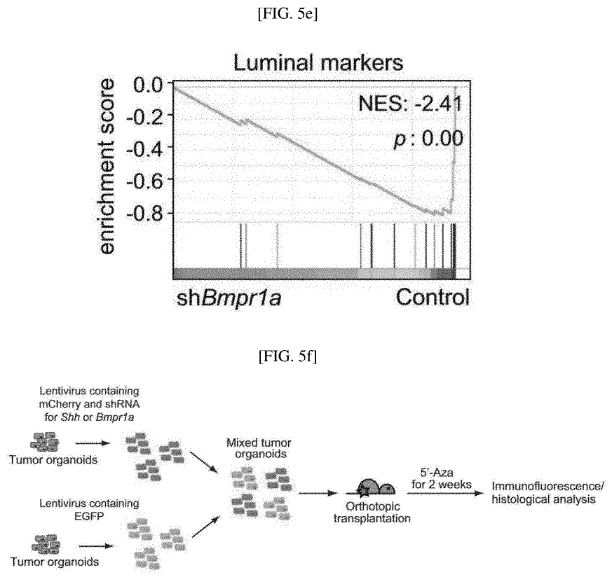

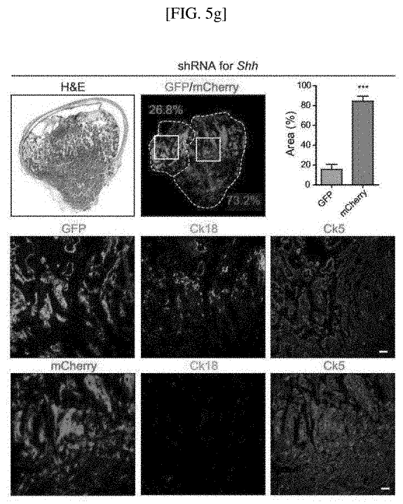

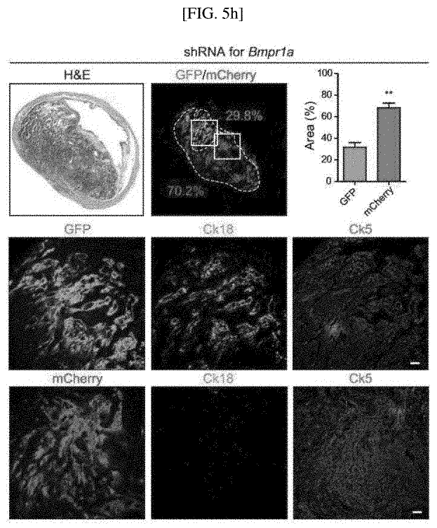

To investigate whether the subtype conversion of bladder cancer cells from a basal subtype to a luminal subtype is mediated by the activated Hh signaling pathway in the tumor cells upon 5′-azacitidine treatment, tumor organoids derived from BBN-induced bladder tumors were infected using a lentivirus containing shRNA targeting Shh or shRNA targeting Bmpr1a, the resulting organoids were injected into the dome of the bladder, and the injected mice (15 mice) were treated with 5′-azacitidine for 2 weeks. The allografts of the mice into which the control tumor organoids (5 mice), and the organoids expressing shRNA targeting Shh (5 mice) or shRNA targeting Bmpr1a (5 mice) were orthotopically injected were stained by H&E staining, and the experimental scheme is shown in FIG. 5A.

The allografts of the mice into which the control tumor organoids, and the organoids expressing shRNA targeting Shh or shRNA targeting Bmpr1a were orthotopically injected were stained by H&E staining, and the results are shown in FIG. 5B (Ck5 is represented in green, and Ck18 is represented in red).

In addition, as shown in FIG. 5C, it was confirmed that the expression of luminal markers was reduced, and more particularly, as compared with the control tumor organoids, in the tumor organoids into which shRNA targeting Shh or Bmpr1a is injected, Upk1a (shRNA targeting Shh: 1.6-fold decrease; shRNA targeting Bmpr1a: 1.5-fold decrease), Upk2 (shRNA targeting Shh: 2-fold decrease; shRNA targeting Bmpr1a: 1.5-fold decrease), Upk3a (shRNA targeting Shh: 2-fold decrease; shRNA targeting Bmpr1a: 1.6-fold decrease) and Krt18 (shRNA targeting Shh: 1.5-fold decrease; shRNA targeting Bmpr1a: 1.5-fold decrease) are reduced (unpaired Student's test (*, p<0.05; **, p<0.01; ***, p<0.001); n=3; the entire experiment was repeated five times).

In addition, using standard luminal signature obtained from previous research, GSEA of tumor allografts expressing shRNA targeting Shh, shRNA targeting Bmpr1a from RNA-Seq data was performed, and the results are shown in FIGS. 5D and 5E.

The tumor organoids were infected with a lentivirus containing EGFP-labeled control shRNA or mCherry-labeled Shh or shRNA targeting Bmpr1a. The same number of each type of the resulting organoids was selected manually, mixed and orthotopically transplanted into nude mice.

Subsequently, the mice (8 mice) were treated with 5′-azacitidine for two weeks, and allografts of the mice into which mixed organoids (four organoids expressing shRNA targeting Shh; four organoids expressing shRNA targeting Bmpr1a) were orthotopically injected were subjected to H&E staining and immunostaining. The experimental scheme is shown in FIG. 5F.

The results of H&E staining and immunostaining with EGFP, mCherry, Ck18 (cyanine, pseudo) and Ck5 (magenta, pseudo) are shown in FIGS. 5G and 5H. The EGFP- or mCherry-positive tumor regions are represented by a dotted line, and each region was measured and quantified using the Image J program (unpaired Student's t test (**, p<0.01; ***, p<0.001). n=4).

These results showed that, when the allografts are treated with 5′-azacitidine, the mCherry-labeled tumors developed to the more aggressive and rapidly growing basal-like subtype, whereas the EGFP-labeled tumors developed to the less aggressive luminal-like subtype in the same microenvironment, indicating the Hh-mediated conversion to a bladder tumor subtype.

5-4. Confirmation of Association of Bmp with Subtype Conversion of Bladder Cancer

To evaluate whether the conversion between a basal-like subtype and a luminal-like subtype further needs Hh-mediated Bmp signaling required for the inhibition of tumor growth, as shown in FIG. 5A, models in which BBN-induced tumor organoids transduced to express shRNA targeting Bmpr1a were orthotopically transplanted into the mouse bladder were established.

As a result, compared with control organoids normally expressing Bmpr1a, it was confirmed that Bmpr1a expression is significantly reduced in the established tumor organoids, and secondary tumors with decreased luminal markers and differentiation to squamous cells are generated in Bmpr1a knock-down tumor organoid grafts in the presence of 5′-azacitidine.

In addition, as shown in FIG. 5E, the RNA-seq expression profiles revealed that the gene signature related to the luminal status was decreased in the tumor expressing shRNA for Bmpr1a, which is consistent with the above results. However, the control tumor showed the standard signature of a luminal-like subtype.

In addition, as shown in FIG. 5F, an experiment in which tumor organoids expressing mCherry-labeled shRNA targeting Bmpr1a were mixed with organoids expressing EGFP-labeled control shRNA, and transplanted into the same in vivo microenvironment was performed.

As a result, as shown in FIG. 5H, it was confirmed that, when allografts are treated with 5′-azacitidine, mCherry-labeled tumors develop into the more aggressive and rapidly growing basal-like subtype, whereas EGFP-labeled tumors develop into the less aggressive luminal-like subtype in the same microenvironment, demonstrating the Hh-mediated conversion of a bladder tumor subtype.

In addition, it was confirmed that, when Bmpr1a is genetically removed by expressing Cre recombinase in BBN-induced tumor organoids derived from Bmpr1a^{flox/flox} mice, consistent with the above-described result, the resulting organoids develop into basal muscle-invasive carcinomas, even with 5′-azacitidine treatment.

Summarizing the above results related to various genetic and pharmacological approaches for Hh and Bmp signal feedback during the growth of bladder cancer, it was confirmed that the conversion between a basal subtype and a luminal subtype depend on the reciprocal signal feedback between tumor cells and the stroma, which involves epigenetically regulated “Shh expression, stromal Hh response induction-Bmp expression and the Bmp response in tumor cells.”

### Example 6

### Confirmation of Induction of Basal Subtype of Human Muscle-Invasive Urothelial Carcinoma

To confirm whether the Hh/BMP signaling feedback between tumor cells and stroma can regulate the growth of a tumor and determine subtypes in human bladder cancer, methylation levels of the promoter region of SHH in human muscle-invasive bladder cancer cell lines J82, T24 and TCC-SUP were measured by bisulfite sequencing analysis.

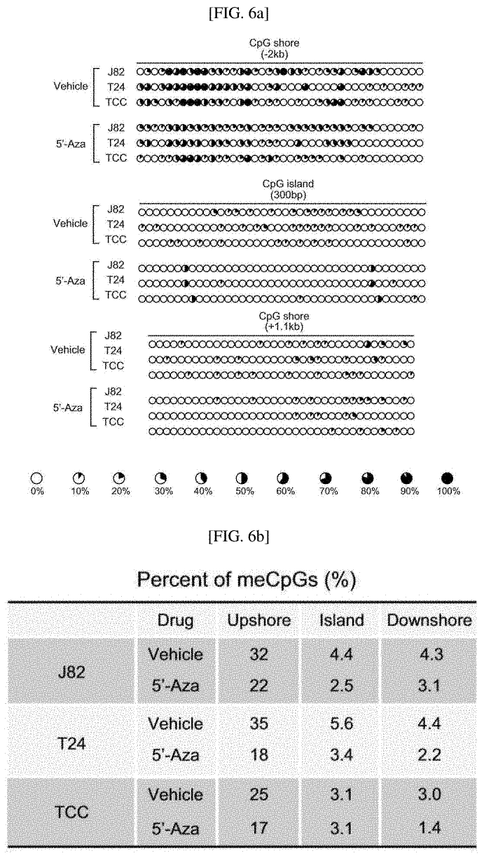

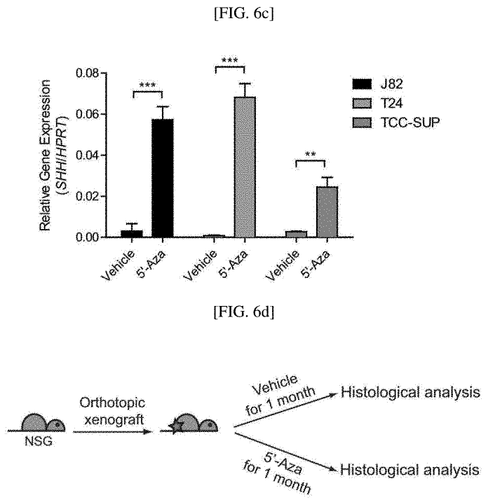

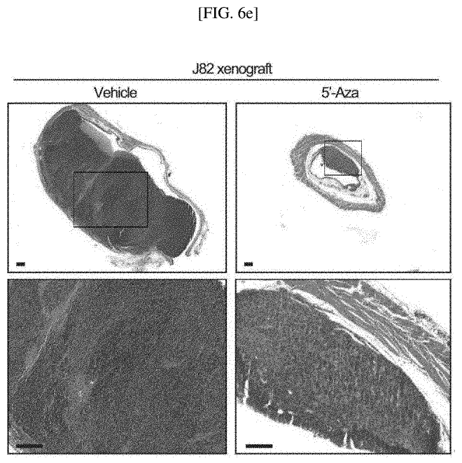

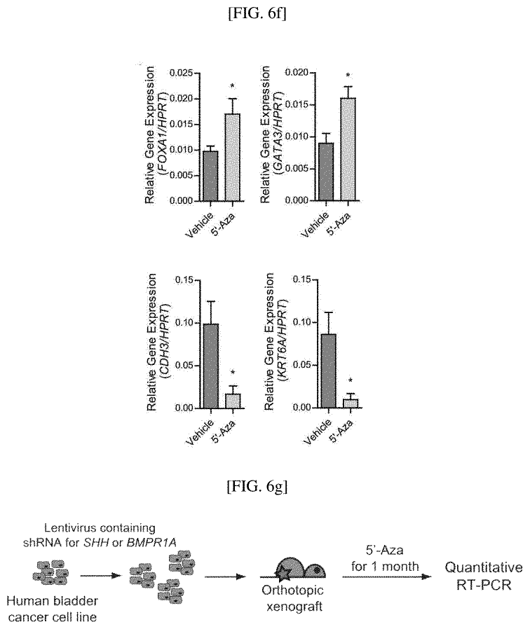

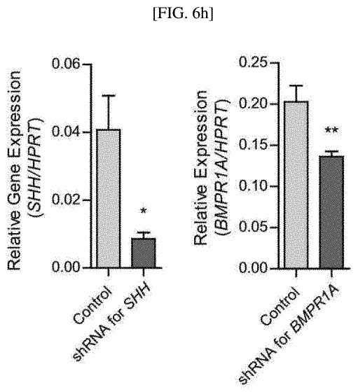

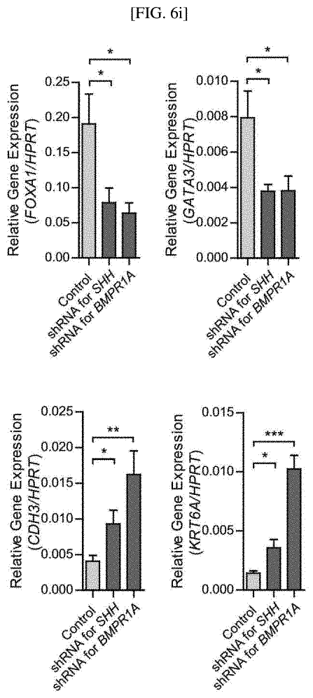

The bisulfite sequencing analysis results for the level of methylation in the promoter region of SHH are as shown in FIG. 6A, and more specifically, the methylation in the CpG shore of the promoter region of the human SHH gene significantly increased, and FIG. 6B shows the summary of the results.

In addition, as shown in FIG. 6C, as compared with the control not treated with 5′-azacitidine, it was confirmed that SHH expression increased in 5′-azacitidine-treated J82, T24 and TCC-SUP, and more particularly, the SHH expression increased 6-fold in J82, 7-fold in T24, and 3-fold in TCC-SUP (unpaired Student's t test (**, p<0.01; ***, p<0.001); n=3; the entire experiment was repeated three times).

To investigate the functional role of SHH expression in the growth of human bladder tumors and the effects of the Hh/BMP signaling feedback between tumors and the stroma on the subtype conversion of human muscle-invasive urothelial carcinoma, xenografts in which J82 cells were orthotopically injected into immunocompromised mice (NOD/SCID/IL2Rgnull) (14 mice) were treated with 5′-azacitidine for one month, and orthotopic xenografts in the mice treated with the vehicle control (7 mice) or 5′-azacitidine (7 mice) were subjected to H&E staining. The experimental scheme is shown in FIG. 6D.

The H&E staining results for sections of the orthotopic xenografts of mice treated with the vehicle control and 5′-azacitidine are shown in FIG. 6E. More specifically, in the vehicle control in which DNA methylation is not inhibited, it was confirmed that tumor cells developed into full-fledged muscle-invasive carcinoma, whereas much smaller cancer lesions were observed in the bladders of the 5′-azacitidine-treated mice, suggesting that the inhibition of DNA methylation inhibits the growth of human bladder tumors.

In addition, as shown in FIG. 6F, compared with the vehicle, in the tumor xenografts of the 5′-azacitidine-treated mice, the expression of luminal markers increased and the expression of basal markers decreased and more particularly, the luminal marker FOXA1 1.8-fold increased, GATA3 1.8-fold increased, the basal marker CDH3 6-fold decreased, and KRT6A 9-fold decreased (unpaired Student's t test (*, p<0.05). n=3, the entire experiment was repeated 6 times).

The results revealed that the 5′-azacitidine-treated xenografts increased the expression of the luminal markers, and exhibited luminal subtype signatures.

In addition, to further confirm the requirements for the Hh/BMP signaling feedback in the subtype conversion of human bladder cancer, the J82 cell line was infected with a lentivirus containing shRNA targeting Shh or Bmpr1a, and the experimental scheme is shown in FIG. 6G.

As a result, as shown in FIG. 6H, it was confirmed that SHH or BMPR1A expression decreased in the tumor xenografts of the mice into which J82 expressing shRNA targeting Shh or Bmpr1a was injected (unpaired Student's t test (*, p<0.05; **, p<0.01)).

In addition, compared with the control J82, it was confirmed that the expression of luminal markers decreased and the expression of basal markers increased in the tumor xenografts of mice into which J82 containing shRNA targeting Shh or Bmpr1a was injected, and more particularly, the expression of the luminal marker FOXA1 (shRNA targeting Shh: 2.5-fold decrease; shRNA targeting Bmpr1a: 3-fold decrease) and GATA3 (shRNA targeting Shh: 2-fold decrease; shRNA targeting Bmpr1a: 2-fold decrease) decreased, and the expression of the basal marker CDH3 (shRNA targeting Shh: 2.3-fold increase; shRNA targeting Bmpr1a: 4-fold increase) and KRT6A (shRNA targeting Shh: 2.5-fold increase; shRNA targeting Bmpr1a: 7.2-fold increase) increased (unpaired Student's t test (*, p<0.05; **, p<0.01; ***, p<0.001)).

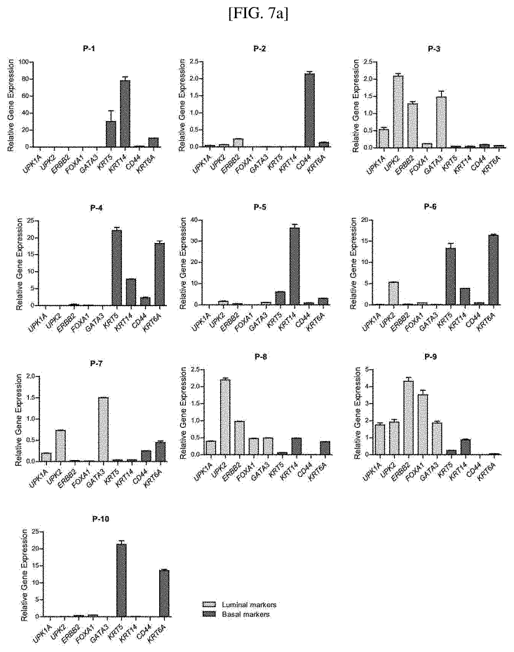

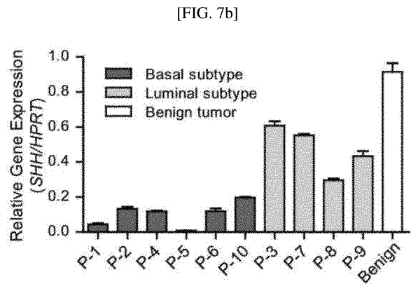

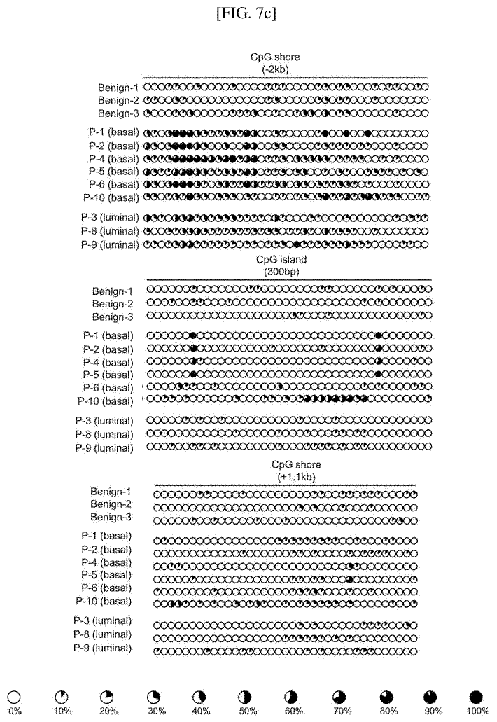

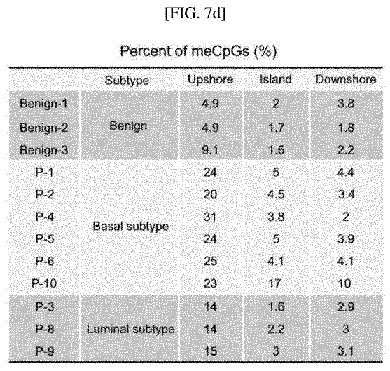

In addition, expression analysis and large-scale transcription analysis of patient-derived urothelial carcinoma were performed. More specifically, the relative expression of basal markers (KRT5, KRT14, CD44 and KRT6A) and luminal markers (UPK1A, UPK2, ERBB2, FOXA1 and GATA3) was analyzed in human invasive urothelial carcinoma derived from 10 patients, and the results are shown in FIG. 7A.

In addition, as shown in FIG. 7B, SHH expression in benign urothelium and relative gene expression levels in two subtypes of invasive urothelial carcinoma were confirmed.

In addition, the methylation levels of the CpG island and CpG shore regions of the human SHH gene in human invasive urothelial carcinoma tissues from patients (3 benign tissues, 6 basal tumors and 3 luminal tumors) were analyzed by bisulfite sequencing, and the results are shown in FIG. 7C, and the summary of the results shown in FIG. 7C is shown in FIG. 7D.

These results are consistent with the results obtained in the mouse model experiment confirming that the increased SHH expression in tumor cells induces the activation of the Hh signaling pathway in the tumor stroma, and the bladder cancer growth is delayed through stromal BMP-induced subtype conversion, and the information on human samples is shown in Table 3.

TABLE 3

| | | | Tumor | | Intra- | Neoajuvant | |
|---|---|---|---|---|---|---|---|
| | | | stage | Tissue | vesical | chemo- | Recur- |
| NO. | Sex | Age | and grade | source | therapy | therapy | rence |
| 1 | M | 65 | T4a (High) | TURB | N | N/A | Y |
| | | | N0 | | | | |
| 2 | M | 61 | T4a (High) | Cystectomy | N | N | N |
| | | | N4 | | | | |
| 3 | M | 56 | T2 (High) | TURB | N | N/A | N |
| 4 | M | 61 | T2 (High) | TURB | N | N/A | N |
| 5 | F | 74 | T2 (High) | TURB | N | N/A | Y |
| 6 | M | 59 | T1 (High) | TURB | BCG | N/A | Y |
| 7 | M | 74 | T1 (High) | TURB | N | N/A | N |
| 8 | M | 62 | T1 (High) | TURB | N | N/A | N |
| | | | N0 | | | | |
| 9 | M | 59 | T3 (High) | TURB | N | N/A | Y |
| 10 | F | 49 | T2 (High) | TURB | N | N/A | N |
| | | | N0 | | | | |

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

1. A method of preventing or treating bladder cancer, comprising:
administering a pharmaceutical composition comprising a methylation inhibitor of the Sonic hedgehog (SHH) gene as an active ingredient into a subject.

2. The method of claim 1, wherein the methylation inhibitor inhibits the methylation of the promoter region of the SHH gene.

3. The method of claim 2, wherein the promoter region is a 2kb-upstream region of a CpG island.

4. The method of claim 1, wherein the methylation inhibitor is 5′-azacitidine.

5. The method of claim 1, wherein the composition increases BMP4 expression.

6. The method of claim 1, wherein the composition inhibits the growth of bladder cancer cells.

7. A method of screening a material for treating bladder cancer, comprising the following steps:
(a) treating a biological sample derived from a subject with a candidate material;
(b) measuring a methylation level of the Sonic hedgehog (SHH) gene in the sample treated with the candidate material; and
(c) selecting the sample as a material for treating bladder cancer when the methylation level of the SHH gene decreases, compared with a control not treated with a candidate material.

8. The method of claim 7, wherein the candidate material is selected from the group consisting of a compound, a microbial culture solution or extract, a natural substance extract, a nucleic acid and a peptide.

9. A method of diagnosing bladder cancer, comprising:
measuring a methylation level of the Sonic hedgehog (SHH) gene.

10. A method of in vitro inducing subtype conversion of bladder cancer cells, comprising:
converting a basal subtype into a luminal subtype.

11. The method of claim 10, wherein the expression of a basal subtype-specific marker decreases, and the expression of a luminal subtype-specific marker increases in the bladder cancer cells.

12. The method of claim 11, wherein the luminal subtype-specific marker is any one or more selected from the group consisting of KRT18, UPK1B, FOXA1, KRT20, GATA3, PPARG, UPK3A, UPK2, UPK1A and Ck18.
